(19) United States
(12) Patent Application Publication
(10) Pub. No.: US 2020/0178851 A1
(43) Pub. Date: Jun. 11, 2020

(54) SYSTEMS AND METHODS FOR TRACKING BODY MOVEMENT

(71) Applicant: Georgia Tech Research Corporation, Atlanta, GA (US)

(72) Inventors: William Singhose, Atlanta, GA (US); Franziska Schlagenhauf, Atlanta, GA (US)

(21) Appl. No.: 16/629,404

(22) PCT Filed: Jul. 10, 2018
(86) PCT No.: PCT/US2018/041468

§ 371 Date: Jan. 8, 2020

Related U.S. Application Data
(60) Provisional application No. 62/530,717, filed on Jul. 10, 2017.

(51) Int. Cl. A61B 5/11; A61B 5/00; G06K 9/00

(52) U.S. Cl. CPC A61B 5/1126; A61B 5/725; G06K 9/00335; A61B 5/1121; A61B 5/1128; A61B 5/1114

(57) ABSTRACT

A system for tracking body movement can comprise a first markerless sensor, a second markerless sensor, a processor, and a memory. The first markerless sensor can be configured to generate a first set of data indicative of positions of at least a portion of a body over a period of time. The second markerless sensor can be configured to generate a second set of data indicative of positions of the at least a portion of the body over the period of time. The memory can comprise logical instructions that, when executed by the processor, cause the processor to generate a third set of data based on the first and second sets of data. The third set of data can be indicative of estimates of at least one of joint positions and joint angles of the at least a portion of the body over the period of time.

### CROSS-REFERENCE TO RELATED APPLICATIONS

This application claims the benefit of U.S. Provisional Application Ser. No. 62/530,717, filed 10 Jul. 2017, which is incorporated herein by reference in its entirety as if fully set forth below.

### TECHNICAL FIELD

The present invention relates generally to motion detection systems and methods. More specifically, the present invention relates to systems and methods for tracking body movement of a subject.

### BACKGROUND

Realistic and accurate human body models are required in many different applications, including, but not limited to medicine, computer graphics, biomechanics, sport science, and the like. A particular application of interest for a human body model is a virtual reality clothing model to evaluate fit and appearance of garments. But to accurately evaluate clothing, a human body model that can produce realistic human motions is helpful.

Clothing fit is one of the most important criterion for customers to evaluate clothing. There is no clear definition of the quality of clothing fit. However, psychological comfort, appearance, and physical dimensional fit contribute to the customer's perceived satisfaction of fit. To assess dimensional fit of a garment, dress forms and 3D body scanning systems are currently used. These methods can reliably evaluate the fit in static poses, but they cannot be used to quickly and accurately assess the quality of fit or change of appearance of a wide range of garments during dynamic poses, e.g., walking, running, jumping, etc.

In recent decades, human body and motion modeling has received increasing attention, with applications in computer vision, virtual reality, and sports science. To date, synthesis of realistic human motions remains a challenge in biomechanics. While clothing simulation is usually accomplished using finite element analysis, evaluation of clothing fit on a real human body performing motions requires a kinematic model capable of predicting realistic human-like motion.

Reliable systems for tracking body movement can also be used to prevent injuries. Work related musculoskeletal disorders (WRMSDs) are a major issue plaguing factory workers, traffic policemen, and others who routinely perform significant upper-body motions. Muscular fatigue is induced due to long working hours, as well as incorrect or sub-optimal motion techniques. Assessment of the range of motion (ROM) of a human joint can yield information about the use, injury, disease, extendability of tendons, ligaments and muscles.

An additional area of interest is the derivation of joint angle trajectories from motion capture data collected from humans in an experimental setting. Such trajectories can, for example, be used to drive a robot through motions that mimic human arm movements. An example for such a robot is shown in FIG. 1, where changes in the shoulder and elbow angles β₁ and β₂ are used to drive the robot.

While many established optical motion capture systems involve multiple high definition cameras and have been proven to be accurate, they are often expensive and infeasible to use outside the confined space in which they are installed. On the other hand, low-cost sensors, such as the Microsoft Kinect sensor, can be non-invasive and used in a wide range of environments. The Kinect has been widely used in the video-gaming industry and can be used to track up to 25 joints of a human skeleton. The sensor provides RGB, depth, and infrared data.

Numerous studies have been presented evaluating the accuracy of skeleton and joint tracking using the first version of the Kinect sensor. Motion capture of upper-body movements using the Kinect compared to a marker-based system has been studied and compared to established optical motion capture methods with respect to applications in ergonomics, rehabilitation, and postural control. Overall, these studies found that the Kinect's precision is less than the optical motion capture system, yet the Kinect has various advantages such as portability, markerless motion capture, and lower cost. To improve the Kinect's motion capture precision, some approaches used additional wearable inertial sensors. With such approaches, more accurate joint angle measurements were obtained.

To further understand the foundation of the present invention, it is helpful to consider the currently available human motion capture tools to assess their capabilities and limitations. The most common approach is to model the human body as a serial multibody system, in which the rigid or flexible bodies (limbs) are connected via joints.

To produce realistic and natural human-like motions, one needs to understand the basic concept of the human structural system and the major movable joints in the real human body. The human musculoskeletal system consists of the bones of the skeleton, cartilage, muscles, ligaments, and tendons. The human skeleton consists of more than 200 bones driven by over 250 muscles, which introduces a great number of degrees of freedom (DoF) into human body models. Different techniques such as physics-based simulation, finite element analysis, and robotic-based methods have been employed with the goal of modeling realistic human motion.

The suitability of an existing model and the derived human-like motions can be evaluated by comparing them with human motion capture systems. The most commonly used motion capture systems are vision-based. These systems can be divided into marker-based and markerless systems. The key difference between these two systems is that marker-based systems require a subject to wear a plurality of reflective markers with the camera/sensor tracking the positions of these markers, but markerless systems require no such reflective markers. For example, while marker-based systems such as OptiTrack or Vicon use multiple cameras to track the positions of reflective markers attached to a human test subject, markerless systems such as the Microsoft Kinect sensor estimate a human pose and joint position based on a depth map acquired with infrared or time-of-flight sensors.

Marker-based systems are widely used and have been established to be fairly accurate. In contrast, markerless systems use position estimation algorithms that introduce error into the measurements. Because current markerless systems have a single camera, only one point of view is available. Occlusion of limbs or movement out of the camera view can cause the pose estimation to fail. While marker-based systems are costly and confined to a certain volumetric workspace, markerless systems are more affordable and can easily be used in many different settings.

Vicon 3D Motion Capture systems involve multiple high definition cameras which are accurate, but expensive, and infeasible to use outside of a highly-controlled laboratory environment such as in shopping malls, airports, boats, roads, etc. On the other hand, the Kinect can be used for human-body motion analysis in a wide variety of settings. The primary differentiating factor between the Kinect and Vicon system is the necessity of retro-reflective markers in the Vicon system. Light from the Vicon cameras is emitted and is reflected from markers in the field of view. This yields the 3D position of each marker. However, the Kinect does not require markers for human-body tracking because a proprietary Microsoft software possesses the ability to track human body joints.

Therefore, there is a desire for improved systems and methods for tracking body movement that overcome the deficiencies of conventional systems. Various embodiments of the present disclosure address this desire.

### SUMMARY

The present disclosure relates to systems and methods for tracking body movement of a subject.

The present invention includes systems for tracking body movement. Systems may comprise a first markerless sensor, a second markerless sensor, a processor, and a memory. The first markerless sensor may be configured to generate a first set of data indicative of positions of at least a portion of a body over a period of time. The second markerless sensor may be configured to generate a second set of data indicative of positions of the at least a portion of the body over the period of time. The memory may comprise logical instructions that, when executed by the processor, cause the processor to generate a third set of data based on the first and second sets of data. The third set of data may be indicative of estimates of at least one of joint positions and joint angles of the at least a portion of the body over the period of time.

In the system discussed above, the memory may further comprise instructions that, when executed by the processor, cause the processor to process the first and second sets of data using a Kalman filter.

In any of the systems discussed above, the Kalman filter may be a linear Kalman filter.

In any of the systems discussed above, the third set of data may be indicative of joint positions of the at least a portion of the body over the period of time.

In any of the systems discussed above, the Kalman filter may be an extended Kalman filter.

In any of the systems discussed above, the third set of data may be indicative of joint angles of the at least a portion of the body over the period of time.

In any of the systems discussed above, the first set of data may include data points indicative of a position for a plurality of predetermined portions of the at least a portion of the body over the period of time, and the second set of data may include data points indicative of a position for the plurality of predetermined portions of the at least a portion of the body over the period of time.

In any of the systems discussed above, for each of the plurality of predetermined portions of the at least a portion of the body, the first and second sets of data may indicate either a specific position for that portion of the at least a portion of the body, an inferred position for that portion of the at least a portion of the body, or no position for that portion of the at least a portion of the body.

In any of the systems discussed above, if the first set of data comprises a first specific position for the first portion of the at least a portion of the body at the specific time and the second set of data comprises a second specific position for the first portion of the at least a portion of the body at the specific time, then the third set of data generated by the processor may comprise a weighted position for the first portion of the at least a portion of the body at the specific time, wherein the weighted position is generated using an average of the first and second specific positions.

In any of the systems discussed above, if only one of the first set of data and the second set of data comprises a specific position for the first portion of the at least a portion of the body at the specific time and the other of the first set of data and the second set of data comprises either an inferred position or no position for the first portion of the at least a portion of the body at the specific time, then the third set of data generated by the processor may comprise a weighted position for the first portion of the at least a portion of the body at the specific time, wherein the weighted position is generated using the specific position in the only one of the first set of data and the second set of data but not the inferred position or the no position in the other of the first set of data and the second set of data.

In any of the systems discussed above, if the first set of data comprises a first inferred position for the first portion of the at least a portion of the body at the specific time and the second set of data comprises a second inferred position for the first portion of the at least a portion of the body at the specific time, then the third set of data generated by the processor may comprise a weighted position for the first portion of the at least a portion of the body at the specific time, wherein the weighted position is generated using an average of the first and second inferred positions.

In any of the systems discussed above, the plurality of predetermined portions of the at least a portion of the body may comprise one or more joints in at least a portion of a human body.

In any of the systems discussed above, the at least a portion of a body may comprise the upper body of a human.

In any of the systems discussed above, the at least a portion of a body may comprise the lower body of a human.

In any of the systems discussed above, the memory may further comprise instructions that, when executed by the processor, cause the processor to transform the positions in at least one of the first set of data and the second set of data into a common coordinate system.

The present invention also includes methods of tracking body movement. A method may comprise generating a first set of data with a first markerless sensor, in which the first set of data may be indicative of positions of at least a portion of a body over a period of time, generating a second set of data with a second markerless sensor, in which the second set of data may be indicative of positions of the at least a portion of the body over the period of time, and processing the first and second sets of data to generate a third set of data, in which the third set of data may be indicative of estimates of at least one of joint positions and joint angles of the at least a portion of the body over the period of time.

The method discussed above may further comprise transforming positions in at least one of the first and second sets of data into a common coordinate system.

In any of the methods discussed above, the first set of data may include data points indicative of a position for a plurality of predetermined portions of the at least a portion of the body over the period of time, and the second set of data may include data points indicative of a position for the plurality of predetermined portions of the at least a portion of the body over the period of time.

In any of the methods discussed above, the plurality of predetermined portions of the at least a portion of the body may comprise one or more joints in at least a portion of a human body.

Any of the methods discussed above can further comprise fusing the first and second sets of data to generate a fourth set of data indicative of weighted positions of the at least a portion of the body over the period of time, in which the weighted positions may be based off of the positions in the first set of data, positions in the second set of data, or a combination thereof.

In any of the methods discussed above, for each of the plurality of predetermined portions of the at least a portion of the body, the first and second sets of data may indicate either a specific position for that portion of the at least a portion of the body, an inferred position for that portion of the at least a portion of the body, or no position for that portion of the at least a portion of the body.

In any of the methods discussed above, if the first set of data comprises a first specific position for the first portion of the at least a portion of the body at the specific time and the second set of data comprises a second specific position for the first portion of the at least a portion of the body at the specific time, then the fourth set of data may comprise a weighted position for the first portion of the at least a portion of the body at the specific time, in which the weighted position is generated using an average of the first and second specific positions.

In any of the methods discussed above, if only one of the first set of data and the second set of data comprises a specific position for the first portion of the at least a portion of the body at the specific time and the other of the first set of data and the second set of data comprises either an inferred position or no position for the first portion of the at least a portion of the body at the specific time, then the fourth set of data may comprise a weighted position for the first portion of the at least a portion of the body at the specific time, in which the weighted position is generated using the specific position in the only one of the first set of data and the second set of data but not the inferred position or no position in the other of the first set of data and the second set of data.

In any of the methods discussed above, if the first set of data comprises a first inferred position for the first portion of the at least a portion of the body at the specific time and the second set of data comprises a second inferred position for the first portion of the at least a portion of the body at the specific time, then the fourth set of data may comprise a weighted position for the first portion of the at least a portion of the body at the specific time, in which the weighted position is generated using an average of the first and second inferred positions.

Any of the methods discussed above may further comprise processing the fourth set of data with a Kalman filter.

In any of the methods discussed above, the Kalman filter may be a linear Kalman filter.

In any of the methods discussed above, processing the fused positions with the linear Kalman filter may generate data indicative of joint positions of the at least a portion of the body over the period of time.

In any of the methods discussed above, the Kalman filter can be an extended Kalman filter.

In any of the methods discussed above, processing the fused positions with the extended Kalman filter may generate data indicative of joint angles of the at least a portion of the body over the period of time.

In any of the methods discussed above, the at least a portion of a body may comprise the upper body of a human.

In any of the methods discussed above, the at least a portion of a body may comprise the lower body of a human.

Any of the methods discussed above may further comprise positioning the first and second markerless sensors.

In any of the methods discussed above, positioning the first and second markerless sensors may comprise positioning the first markerless sensor in a fixed position relative to the body, positioning the second markerless sensor in a temporary position relative to the body, and iteratively altering the position of the second markerless sensor relative to the body by moving the second markerless sensor around the body and checking the accuracy of the estimates of at least one of joint positions and joint angles of the at least a portion of the body over the period of time in the third set of data to determine an optimal position for the second markerless sensor.

In any of the methods discussed above, positioning the first and second markerless sensors may comprise positioning the first and second markerless sensors adjacent to each other relative to the body, and iteratively altering the position of both the first and second markerless sensors relative to the body by moving both the first and second markerless sensors around the body and checking the accuracy of the estimates of at least one of joint positions and joint angles of the at least a portion of the body over the period of time in the third set of data to determine an optimal position for the first and second markerless sensors.

In any of the methods discussed above, the accuracy may be determined based on a difference between the estimates in the third set of data and estimates determined using a marker-based system.

In any of the methods discussed above, the accuracy may be determined based on a number of inferred positions and no positions in the first and second sets of data.

These and other aspects of the present disclosure are described in the Detailed Description below and the accompanying figures. Other aspects and features of embodiments of the present disclosure will become apparent to those of ordinary skill in the art upon reviewing the following description of specific, example embodiments of the present disclosure in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the disclosure discussed herein. In similar fashion, while example embodiments may be discussed below as device, system, or method embodiments, it is to be understood that such example embodiments can be implemented in various devices, systems, and methods of the present disclosure.

### BRIEF DESCRIPTION OF THE DRAWINGS

The following Detailed Description is better understood when read in conjunction with the appended drawings. For the purposes of illustration, there is shown in the drawings example embodiments, but the subject matter is not limited to the specific elements and instrumentalities disclosed.

FIG. 1 provides an illustration of a prior art robotic joint.

FIG. 2 illustrates Denavit-Hartenberg parameters and link frames, in accordance with an exemplary embodiment of the present invention.

FIG. 3 shows the locations of joints in a torso model, in accordance with an exemplary embodiment of the present invention.

FIG. 4 shows the coordinate frames assigned to the joints and the joint angles, in accordance with exemplary embodiment of the present invention.

FIG. 5 shows the location of joints in an upper body model, in accordance with an exemplary embodiment of the present invention.

FIG. 6 shows the coordinate frames and joint angles for a left arm model, in accordance with an exemplary embodiment of the present invention.

FIG. 7 shows body segment lengths for an upper body model, in accordance with an exemplary embodiment of the present invention.

FIG. 8 shows the workflow of a proposed motion tracking system, in accordance with an exemplary embodiment of the present invention.

FIGS. 9A-B illustrate methods of positioning sensors, in accordance with exemplary embodiments of the present invention.

FIG. 10 illustrates sensor positions of a motion tracking system, in accordance with exemplary embodiments of the present invention.

FIG. 11 provides an algorithm for implementing a linear Kalman filter, in accordance with exemplary embodiments of the present invention.

FIG. 12 provides an algorithm for implementing an extended Kalman filter, in accordance with exemplary embodiments of the present invention.

FIG. 13 shows the locations of markers for a full body Plug-in-Gait model.

FIGS. 14A-B show a subject standing in the T-Pose while facing the Dual-Kinect setup.

FIGS. 15A-B show a test subject wearing a motion capture suit with the attached markers.

FIGS. 16-24 provide plots of experimental testing results, in accordance with exemplary embodiments of the present invention.

FIGS. 25 and 26A-F provide illustrations of GUI's illustrating experimental testing results, in accordance with exemplary embodiments of the present invention.

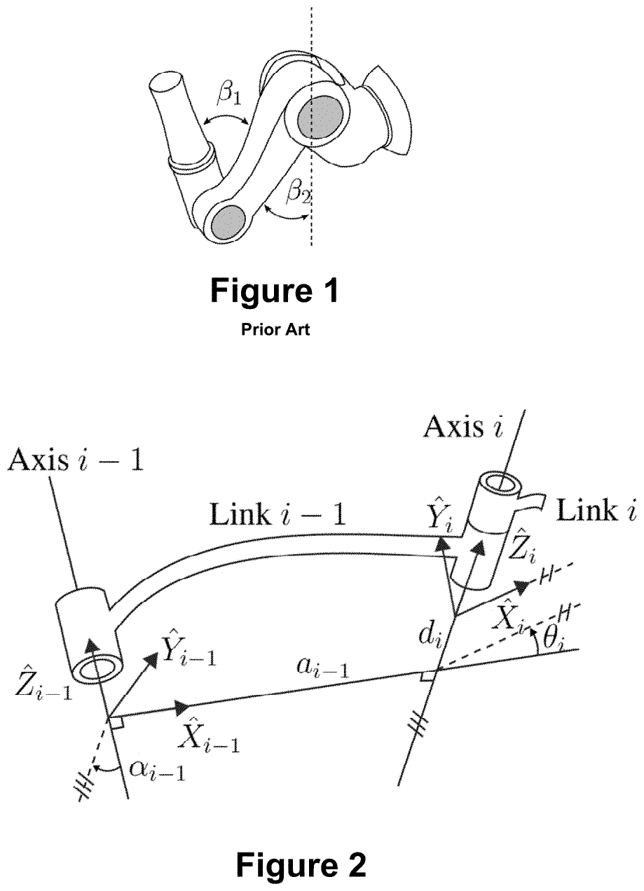

### DETAILED DESCRIPTION

To facilitate an understanding of the principles and features of the present disclosure, various illustrative embodiments are explained below. To simplify and clarify explanation, the disclosed technology is described below as applied to tracking movement of the upper body in a human subject using two sensors. One skilled in the art will recognize, however, that the disclosed technology is not so limited. Rather, various embodiments of the present invention can also be used to track movement of other portions of the human body (including portions of the upper and lower body of a human object), the human body as a whole, and even various portions of non-human objects.

The components, steps, and materials described hereinafter as making up various elements of the disclosed technology are intended to be illustrative and not restrictive. Many suitable components, steps, and materials that would perform the same or similar functions as the components, steps, and materials described herein are intended to be embraced within the scope of the disclosed technology. Such other components, steps, and materials not described herein can include, but are not limited to, similar components or steps that are developed after development of the disclosed technology.

The human upper body can be modeled as a series of links that are connected by joints. In order to employ a robotics-based framework, the anatomical joints can be decomposed into a series of revolute, single DoF joints.

Key Joints and Degrees of Freedom

In order to develop a kinematic model, it is helpful to understand the major movable joints of the real human body. The upper body can be divided into a torso segment, a head segment including the neck, and the arms. In the model discussed below, the head segment is neglected in the modeling process. Persons of ordinary skill in the art, however, would understand that various embodiments of the present invention can further encompass modeling the head segment (or any other portions of the body).

Motion of the torso segment arises mainly from the vertebral column or spine, which consists of multiple discs. To sufficiently model the mobility of the spine, but at the same time limit the degrees of freedom, the spine can be divided into three regions: a lower region (sacrum and coccyx), a middle region (chest or thoracic region), and an upper region (located approximately at the sternum). The movable parts in each of these regions can be modeled as a 3-DoF universal joint, enabling 3-axis motion.

The major joints of the human arm are located in the shoulder, elbow, and wrist. Shoulder motion is achieved through the shoulder complex, which consists of 20 muscles, three functional joints and three bony articulations. However, the term “shoulder joint” usually refers to only one particular joint, the glenohumeral joint, which is a ball-and-socket-type joint. Usually only the shoulder joint is considered in models of anthropometric arms. It is commonly modeled as a 3-DoF universal joint, which is sufficient to enable 3-axis motion of the upper arm. The elbow and wrist joints are each modeled with two DoF.

Using a robotics-based approach to modeling the human upper body, the rotation of each body segment can be defined by joint angles θᵢ, i=1 . . . n, where n is the number of single-DoF joints in the complete model. The orientation and position of the links in the kinematic chain can then be expressed using Denavit-Hartenberg parameters.

Denavit-Hartenberg Parameters

In order to describe the spatial configuration of a serial robot, Denavit-Hartenberg (DH) parameters are commonly used. Each joint i is assigned a frame O with location p. FIG. 2 shows the relation between DH parameters and frames i−1 and i for a segment of a general manipulator, in accordance with an exemplary embodiment of the present invention. dᵢ is the distance from Oᵢ₋₁ to Oᵢ, measured along Zᵢ. aᵢ is the distance from Zᵢ to Zᵢ₊₁, measured along Xᵢ. θᵢ is the joint angle between Xᵢ₋₁ and Xᵢ measured about Zᵢ. aᵢ is the angle between Zᵢ and Zᵢ₊₁, measured about Xᵢ. A 4 X 4 homogeneous transformation matrix (shown in Equation 1) can be used to transform frame i to i+1:

$${T}_{i}^{i + 1} = [ \begin{matrix}cos {θ}_{i} & - sin {θ}_{i} cos {α}_{i} & sin {θ}_{i} sin {α}_{i} & {a}_{i} cos {θ}_{i} \\ sin {θ}_{i} & cos {θ}_{i} cos {α}_{i} & - cos {θ}_{i} sin {α}_{i} & {a}_{i} sin {θ}_{i} \\ 0 & sin {α}_{i} & cos {α}_{i} & {d}_{i} \\ 0 & 6 & 0 & 1\end{matrix} ] \qquad Equation 1$$

with joint angle θᵢ, link twist aᵢ, link length aᵢ and link offset dᵢ.

Multiple options for the placement of the coordinate frames generally exist. Below, the major anatomical joints of the upper body are decomposed into single-DoF revolute joints and the DH parameters for the torso and arm model are derived.

Torso Model

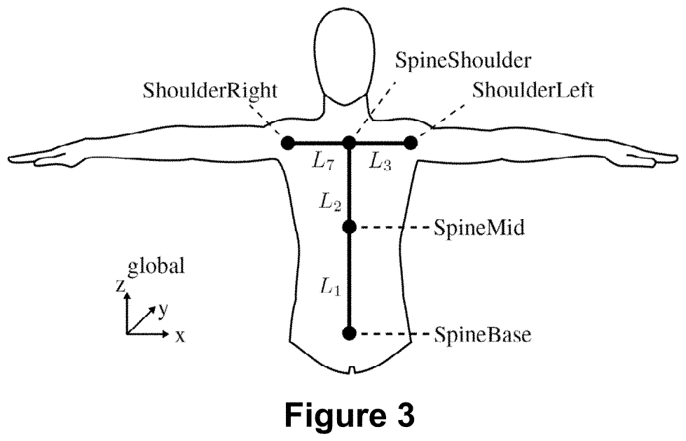

The torso can be modeled as a tree-structured chain composed of four rigid links: one link from the base of the spine to the spine midpoint, one link from the spine midpoint to the spine at the shoulder, approximately located at the sternum, and two links connecting spine at the shoulder to the left and right shoulder. The corresponding joints in the torso model will be referenced to as “SpineBase,” “SpineMid,” and “SpineShoulder,” with the “SpineShoulder” connecting to the “ShoulderLeft” and “ShoulderRight.” FIG. 3 shows the locations of these joints in the human body, in accordance with an exemplary embodiment of the present invention.

Because this embodiment only considers movement in the upper body, the base of the spine is assumed to be fixed in space. The lower spine region can be considered as a universal joint that can be modeled as three independent, single-DoF revolute joints with intersecting orthogonal axes. The corresponding joint angles are θ₁, θ₂, and θ₃. The same approach is taken to model motion in the mid region of the spine. The “SpineMid” enables the torso to rotate and bend about three axes with joint angles θ₄, θ₅, and θ₆. At the “SpineShoulder,” the kinematic chain is split into two branches, allowing for independent motion of both shoulder joints relative to the sternum. For each branch, the shoulder joint is modeled as three independent, single-DoF revolute joints. The link connecting the “SpineShoulder” with the “ShoulderLeft” can be moved with joint angles θ₇, θ₈, and θ₉, while the right link can be moved with θ₁₀, θ₁₁, and θ₁₂, respectively.

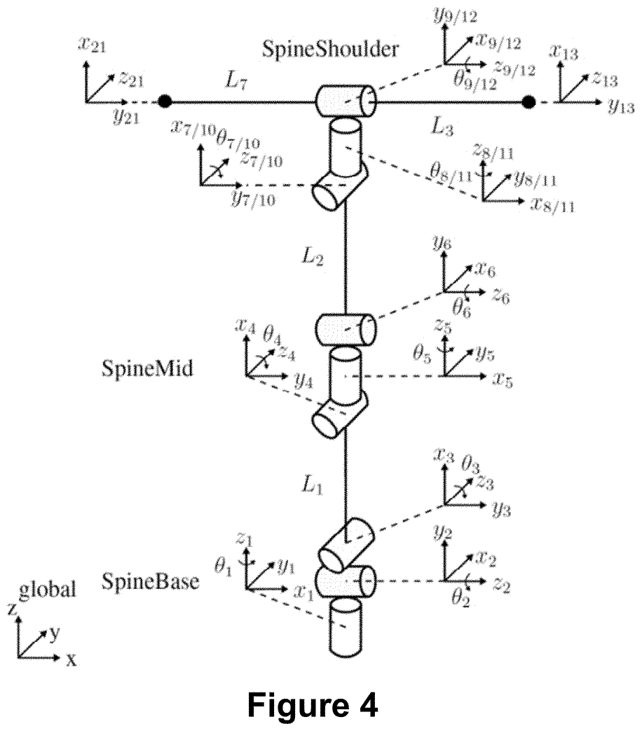

In summary, the complete torso model can comprise four rigid links, interconnected by 12 single-DoF revolute joints. Using the DH conventions, coordinate systems and corresponding DH parameters can be assigned to each joint. FIG. 4 shows the coordinate frames assigned to the joints and the joint angles, in accordance with exemplary embodiment of the present invention. The corresponding DH parameters for the torso model are listed below in Table 2. Provided the link lengths L₁, L₂, L₃ and L₇, and the 12 joint angles θ₁, θ₂, . . . , θ₁₂, the spatial configuration of the torso model can be completely defined.

TABLE 2
DH Parameters for Torso Model

| i | Joint | θᵢ | dᵢ | aᵢ | aᵢ |
|---|---|---|---|---|---|
| 1 | SpineBase Z | θ₁ + π/2 | 0 | 0 | π/2 |
| 2 | SpineBase X | θ₂ + π/2 | 0 | 0 | π/2 |
| 3 | SpineBase Y | θ₃ | 0 | L₁ | 0 |
| 4 | SpineMid Y | θ₄ + π/2 | 0 | 0 | π/2 |
| 5 | SpineMid Z | θ₅ + π/2 | 0 | 0 | π/2 |
| 6 | SpineMid X | θ₆ + π/2 | 0 | L₂ | π/2 |
| 7 | SpineShoulder Y (left) | θ₇ + π/2 | 0 | 0 | π/2 |
| 8 | SpineShoulder Z (left) | θ₈ + π/2 | 0 | 0 | π/2 |
| 9 | SpineShoulder X (left) | θ₉ + π/2 | L₃ | 0 | π/2 |
| 10 | SpineShoulder Y (right) | θ₁₀ + π/2 | 0 | 0 | π/2 |
| 11 | SpineShoulder Z (right) | θ₁₁ + π/2 | 0 | 0 | π/2 |
| 12 | SpineShoulder X (right) | θ₁₂ + π/2 | −L₇ | 0 | π/2 |

Arm Model

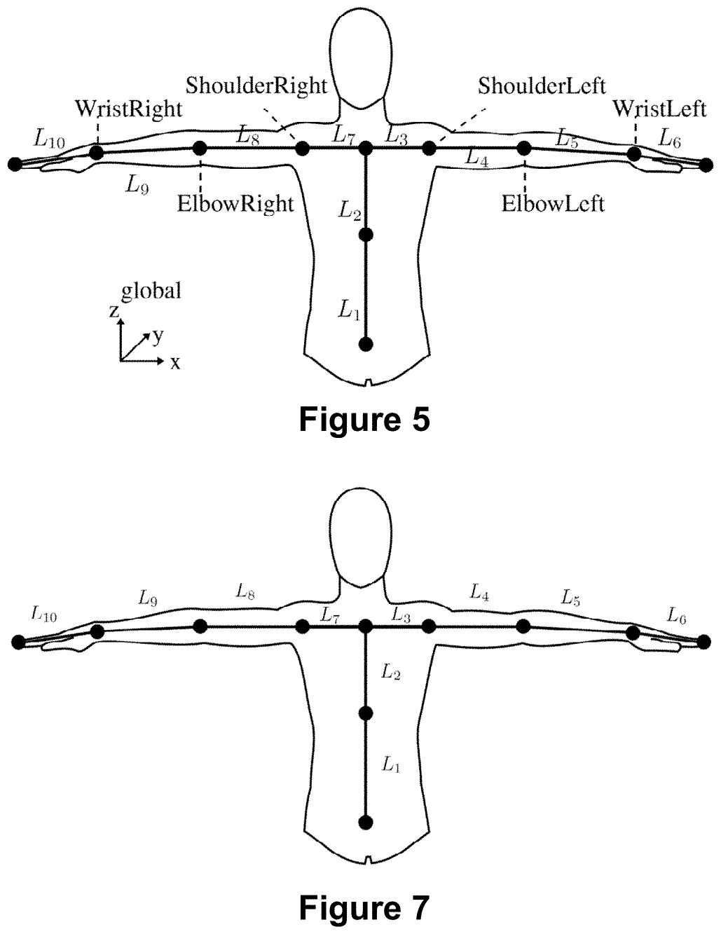

Each arm can be modeled as a serial kinematic chain comprising three links: one link from the shoulder joint to the elbow joint, one from elbow to the wrist, and one link from the wrist to the tip of the hand. The corresponding link lengths can be defined as L₄, L₅, and L₆ for the left arm, and L₈, L₉, and L₁₀ for the right arm. The joints can be referenced to as “ShoulderLeft,” “ElbowLeft,” “WristLeft,” “ShoulderRight,” “ElbowRight,” and “WristRight,” respectively. FIG. 5 shows the location of these joints in the body, in accordance with an exemplary embodiment of the present invention. The anatomical shoulder joint can be modeled as a universal joint, providing three DoFs for the rotation of the upper arm. The left (right) shoulder joint can therefore be modeled as three independent, single-DoF revolute joints with intersecting orthogonal axes with joint angles θ₁₃, θ₁₄, and θ₁₅ (right: θ₂₀, θ₂₁, and θ₂₂). The elbow can be modeled as two single-DoF revolute joints with joint angles θ₁₆ and θ₁₇ (right: θ₂₃ and θ₂₄). The wrist can be modeled as two single-DoF revolute joints with joint angles θ₁₈ and θ₁₉ (right: θ₂₅ and θ₂₆).

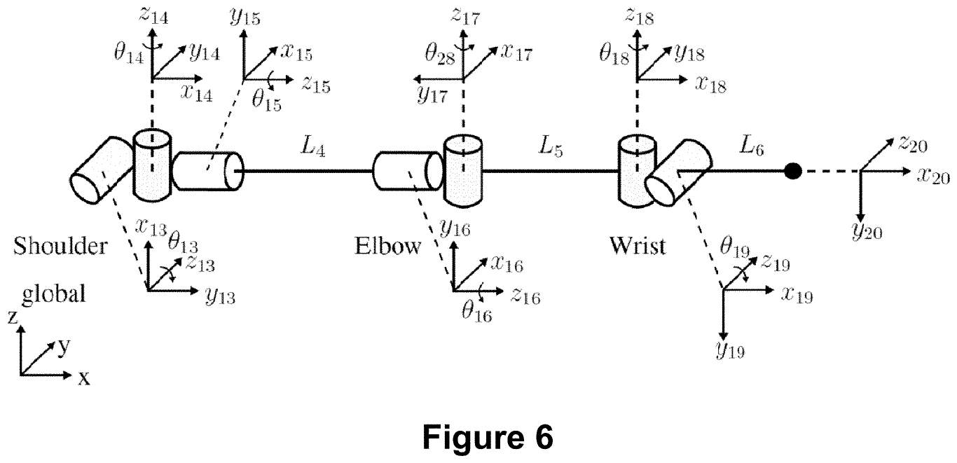

FIG. 6 shows the coordinate frames and joint angles for the left arm model, in accordance with an exemplary embodiment of the present invention. The corresponding DH parameters for the left and right arm model are listed in Table 3. Adding up the DoF for the shoulder, elbow, and wrist, each arm model has seven DoFs.

TABLE 3
DH Parameters for the Left and Right Arm Model

| i | Joint | θᵢ | dᵢ | aᵢ | aᵢ |
|---|---|---|---|---|---|
| 13 | ShoulderLeft Y | θ₁₃ + π/2 | 0 | 0 | π/2 |
| 14 | ShoulderLeft Z | θ₁₄ + π/2 | 0 | 0 | π/2 |
| 15 | ShoulderLeft X | θ₁₅ | L₄ | 0 | 0 |
| 16 | ElbowLeft X | θ₁₆ | 0 | 0 | −π/2 |
| 17 | ElbowLeft Z | θ₁₇ − π/2 | 0 | L₅ | 0 |
| 18 | WristLeft Z | θ₁₈ | 0 | 0 | −π/2 |
| 19 | WristLeft Y | θ₁₉ | 0 | L₆ | 0 |
| 20 | ShoulderRight Y | θ₂₀ + π/2 | 0 | 0 | π/2 |
| 21 | ShoulderRight Z | θ₂₁ + π/2 | 0 | 0 | π/2 |
| 22 | ShoulderRight X | θ₂₂ | −L₈ | 0 | 0 |
| 23 | ElbowRight X | θ₂₃ | 0 | 0 | −π/2 |
| 24 | ElbowRight Z | θ₂₄ − π/2 | 0 | −L₉ | 0 |
| 25 | WristRight Z | θ₂₅ | 0 | 0 | −π/2 |
| 26 | WristRight Y | θ₂₆ | 0 | −L₁₀ | 0 |

Because only six DoFs are used to define the position and orientation of the end-effector (tip of the hand), it follows that the human arm model is redundant. Redundancy is defined as the number of joints exceeding the output degrees of freedom. For the human arm, this redundancy can be observed by, first, fixing the positions of the shoulder and wrist in space. Then allow the elbow to move without moving the shoulder or wrist position. Combining the torso and arm model further increases redundancy, making the upper body model a highly redundant system.

Offsets in the joint angles θᵢ can be introduced to place the upper body model in the rest position with both arms fully extended to the sides (=T-Pose), shown in FIGS. 3 and 5 when θᵢ=0 for i=1, . . . 26. The body segment lengths for the upper body model are shown in FIG. 7. Table 4 lists the names of the corresponding segments. Table 5 gives an overview of the biomechanical motions provided by each joint angle.

TABLE 4
Body Segment Lengths

| Lᵢ | Body Segment | Lᵢ | Body Segment |
|---|---|---|---|
| L₁ | Lower Torso | L₆ | Left Palm |
| L₂ | Upper Torso | L₇ | Right Clavicle |
| L₃ | Left Clavicle | L₈ | Right Upper Arm |
| L₄ | Left Upper Arm | L₉ | Right Forearm |
| L₅ | Left Forearm | L₁₀ | Right Palm |

TABLE 5
Joint Motion Definitions

| i | Joint Name | Joint Angle | Motion |
|---|---|---|---|
| 1 | SpineBase Z | θ₁ | Lumbar Flexion/Extension |
| 2 | SpineBase Y | θ₂ | Lumbar Lateral |
| 3 | SpineBase X | θ₃ | Lumbar Rotation |
| 4 | SpineMid Y | θ₄ | Thoracic Lateral |
| 5 | SpineMid Z | θ₅ | Thoracic Flexion/Extension |
| 6 | SpineMid X | θ₆ | Thorax Rotation |
| 7 | SpineShoulder (left) Y | θ₇ | Left Clavicle Elevation/Depression |
| 8 | SpineShoulder (left) Z | θ₈ | Left Clavicle Flexion/Extension |
| 9 | SpineShoulder (left) X | θ₉ | Left Clavicle Rotation |
| 10 | SpineShoulder (right) Y | θ₁₀ | Right Clavicle Elevation/Depression |
| 11 | SpineShoulder (right) Z | θ₁₁ | Right Clavicle Flexion/Extension |
| 12 | SpineShoulder (right) X | θ₁₂ | Right Clavicle Rotation |
| 13 | ShoulderLeft Y | θ₁₃ | Left Shoulder Abduction/Adduction |
| 14 | ShoulderLeft Z | θ₁₄ | Left Shoulder Flexion/Extension |
| 15 | ShoulderLeft X | θ₁₅ | Left Shoulder Medial/Lateral Rotation |
| 16 | ElbowLeft X | θ₁₆ | Left Elbow Pronation/Supination |
| 17 | ElbowLeft Z | θ₁₇ | Left Elbow Flexion/Extension |
| 18 | WristLeft Z | θ₁₈ | Left Wrist Radial/Ulnar Deviation |
| 19 | WristLeft Y | θ₁₉ | Left Wrist Flexion/Extension |
| 20 | ShoulderRight Y | θ₂₀ | Right Shoulder Adduction/Abduction |
| 21 | ShoulderRight Z | θ₂₁ | Right Shoulder Flexion/Extension |
| 22 | ShoulderRight X | θ₂₂ | Right Shoulder Medial/Lateral Rotation |
| 23 | ElbowRight X | θ₂₃ | Right Elbow Pronation/Supination |
| 24 | ElbowRight Z | θ₂₄ | Right Elbow Flexion/Extension |
| 25 | WristRight Z | θ₂₅ | Right Wrist Radial/Ulnar Deviation |
| 26 | WristRight Y | θ₂₆ | Right Wrist Flexion/Extension |

Forward Kinematics

Given the values for all link lengths and joint angles, the position and orientation of the joints up to the end-effector (tip of the hand) can be expressed in the base frame. It can be calculated using the transformation matrices with the DH-Parameters of the kinematic model listed in Tables 2 and 3. These kinematic equations state the forward kinematics of the upper body model. Using the joint angles as generalized coordinates in the joint vector q=[θ₁ . . . θ₂₆]^{T}, the pose of the serial manipulator can be calculated as a function of the joint angles:

x=f(q) Equation 2:

The position p and orientation [n s o] of the i^{th} joint, expressed in the base frame, can be calculated by multiplication of the transformation matrices:

$$[ \begin{matrix}n & s & 0 & p \\ 0 & 0 & 0 & 1\end{matrix} ] = {T}_{0}^{1} {T}_{1}^{2} … {T}_{i - 1}^{i} \qquad Equation 3$$

Inverse Kinematics

The inverse kinematics of a system can be generally used to calculate joint angles q based on a given position and orientation of an end-effector x:

q=f⁻¹(x) Equation 4:

Solving the inverse kinematics problem is not as straight-forward as calculating the forward kinematics. Due to the kinematic equations being nonlinear, their solution is not always obtainable in closed form. Because the developed upper body model can be a highly redundant system, the conventional inverse kinematics for a closed-form solution can be difficult to apply. Accordingly, instead of calculating a closed-form solution, some embodiments of the present invention use a Jacobian-based approach. The Jacobian can provide a mapping between joint angle velocities q and Cartesian velocities x

\dot{x}=J(q)\dot{q}, Equation 5:

where J is the Jacobian matrix ∂f/∂q.

State Estimation Methods for Joint Tracking

Considering a state-space representation, the system model can describe the dynamics of the system, or in this case how the links of the upper body model move in time. The observation model can describe the relationship between the states and measurements. In some embodiments of the present invention, a linear Kalman filter and an extended Kalman filter can be used for joint tracking.

State Space Models: If it can be assumed that a tracked object, such as a joint of the human body, is executing linear motion, the linear Kalman filter can be used to estimate the states of a system. Below, two commonly used examples of discrete-time state space models describing the motion of an object in 3D space are presented. For the sake of simplicity, the equations are derived to track a single joint's position. The models presented here are later used with the linear Kalman filter algorithm.

Zero Velocity Model: Assuming the velocity of the joint to be zero, the state vector for a problem with three spacial dimensions is given by s=[x y z]^{T} and the state space model is given by:

sₖ₊₁=Asₖ+ωₖ Equation 6:

zₖ=Csₖ+vₖ, Equation 7:

where the state transition matrix is given by

$$A = [ \begin{matrix}1 & 0 & 0 \\ 0 & 1 & 0 \\ 0 & 0 & 1\end{matrix} ] \qquad Equation 8$$

The observation matrix C takes into account the observed coordinates of the joint position and is given by:

$$C = [ \begin{matrix}1 & 0 & 0 \\ 0 & 1 & 0 \\ 0 & 0 & 1\end{matrix} ] \qquad Equation 9$$

Constant Velocity Model: Another approach is to model the joint to be moving with constant velocity and taking into account the joint velocities as states. For a 3D problem, the state space vector becomes 6-dimensional: s=[x y z \dot{x} \dot{y} ż]^{T}. The state space model can have the same form as in the zero velocity model in Equations 6 and 7, with the state transition matrix given by

$$A = [ \begin{matrix}1 & 0 & 0 & Δ t & 0 & 0 \\ 0 & 1 & 0 & 0 & Δ t & 0 \\ 0 & 0 & 1 & 0 & 0 & Δ t \\ 0 & 0 & 0 & 1 & 0 & 0 \\ 0 & 0 & 0 & 0 & 1 & 0 \\ 0 & 0 & 0 & 0 & 0 & 1\end{matrix} ] \qquad Equation 10$$

where Δt is the sampling time. If only the positions, and not the velocities are observed, the observation matrix is given by

$$C = [ \begin{matrix}1 & 0 & 0 & 0 & 0 & 0 \\ 0 & 1 & 0 & 0 & 0 & 0 \\ 0 & 0 & 1 & 0 & 0 & 0\end{matrix} ] \qquad Equation 11$$

Linear Kalman Filter

The Kalman filter is a recursive algorithm used to estimate a set of unknown parameters (in this case the states s) based on a set of measurements z. It uses a prediction and an update step. The linear Kalman filter provides an optimal solution to the linear quadratic estimation problem. Assume the system and measurement models are linear and given by:

sₖ₊₁=Fₖsₖ+Bₖuₖ+wₖ Equation 12:

zₖ=Hₖsₖ+vₖ Equation 13:

Fₖ is the state transition matrix, Bₖ is the input matrix, Hₖ is the observation matrix, wₖ is the process noise, and vₖ is the measurement noise. It can be assumed that the process and measurement noises are zero-mean, Gaussian noise vectors with covariance matrices Qₖ and Rₖ, i.e. w˜N(0, Qₖ) and v˜N(0, Rₖ). The covariance matrices are:

Qₖ=E(wₖwₖ^{T}) Equation 14:

Rₖ=E(vₖvₖ^{T}) Equation 15:

Consider that at time k the state estimate ŝ_{k|k} and error covariance matrix P_{k|k} are known and contain the information provided by all previous measurements. In the prediction step of the Kalman filter, these quantities can be propagated forward in time using:

ŝ_{k|k-1}=Fₖŝ_{k-1|k-1}+Bₖuₖ Equation 16:

P_{k|k-1}=FₖP_{k-1|k-1}F^{T}+Qₖ Equation 17:

If a new measurement is available, then the update step can be performed:

yₖ=zₖ−Hₖŝ_{k|k-1} Equation 18:

ŝ_{k|k}=ŝ_{k|k-1}+Kₖyₖ Equation 19:

P_{k|k}=(I−KₖHₖ)P_{k|k-1} Equation 20:

Equation 18 is a measure of the error between the measurement zₖ and the current state estimate mapped into the measurement space. This measure is weighted by the Kalman gain:

Kₖ=P_{k|k-1}Hₖ^{T}(HₖP_{k|k-1}Hₖ^{T}+Rₖ)⁻¹. Equation 21:

Extended Kalman Filter

While the linear Kalman filter can be used for linear systems, the Extended Kalman Filter (EKF) extends the algorithm to work on nonlinear systems. Consider a nonlinear model:

sₖ₊₁f(sₖ,uₖ)+wₖ Equation 22:

zₖ=h(sₖ)+vₖ Equation 23:

The true state and measurement vectors can be approximated by linearizing the system about the current state estimate using a first-order Taylor series expansion:

sₖ₊₁≈f(ŝₖ)+Fₖ(sₖ−ŝₖ) Equation 24:

zₖ≈h(ŝₖ)+Hₖ(sₖ−ŝₖ) Equation 25:

Fₖ and Hₖ are the Jacobians of the system and measurement models, evaluated at the current state estimate:

$$\begin{matrix}{F}_{k} = \frac{\partial f}{\partial s} {\vert}_{s = {\hat{s}}_{k}} & Equation 26 \\ {H}_{k} = \frac{\partial h}{\partial s} {\vert}_{s = {\hat{s}}_{k}} & Equation 27\end{matrix}$$

After linearizing the system, the standard Kalman Filter can be applied. It should be noted that contrary to the linear Kalman filter, the EKF is not optimal. The filter is also still subject to the assumption of Gaussian noise for the process and measurement.

Dual Sensor Motion Capture

Below, an exemplary embodiment of the present invention is disclosed, which employs two Kinect camera sensors for real-time motion capture measurements. To demonstrate the performance of this system, it is used to track a human test subject conducting a set of three different motions (“two-handed wave,” “slow-down signal,” and “torso twist”). Further testing with loose-fitting clothes demonstrates the robustness of this embodiment. During these tests, the test subject conducted motions commonly performed to test fit of garments, such as the torso twist, calf extensions, and squats.

The dual-Kinect system uses Kalman filters, such as those discussed above, to fuse the two data streams from each sensor and improve joint tracking. For analyzing the results in detail, a script that records the joint position estimates from both Kinect sensors was implemented. To evaluate the tracking performance, data was concurrently obtained with a Vicon motion capture system, which employed reflective markers.

The recorded data was used to analyze the joint position tracking performance for different filter parameters for a linear Kalman filter (LKF) and for the Extended Kalman filter (EKF) based on the kinematic human upper body model discussed previously. Results from human motion capture experiments with the inventive dual-Kinect system and both filters are compared to marker-based motion capture data collected with a Vicon system.

Dual-Kinect Motion Capture Process

An embodiment of the present invention comprising two markerless sensors will now be described. It should be understood, however, that the present invention is not limited to use of only two markerless sensors. Rather, various embodiments of the present invention can employ three or more markerless sensors. Additionally, some embodiments can employ two or more markerless sensors in conjunction with one or more marker-based sensors.

As discussed in more detail below, exemplary embodiments of the present invention provide systems for tracking movement of an object. A system may comprise a first markerless sensor, a second markerless sensor, a processor, and a memory. For purposes of illustration wherein, the markerless sensors can be Microsoft Kinect sensors. The present invention, however, is not limited to any particular markerless sensor. Rather, the markerless sensors can be many markerless different sensors. Additionally, the present invention is not limited to use of only two markerless sensors. Rather, the present invention includes embodiments using three or more markerless sensors. The present invention also does not necessarily exclude the use of marker-based sensors. For example, some embodiments of the present invention can employ marker-based sensors or combinations of markerless and marker-based sensors.

The first markerless sensor may be configured to generate a first set of data indicative of positions of at least a portion of a body over a period of time. The second markerless sensor may be configured to generate a second set of data indicative of positions of the at least a portion of the body over the period of time. The data sets generated by the markerless sensors can include various data regarding the objects sensed (e.g., portions of a body), including, but not limited to, positions of various features, color (e.g., RGB), infrared data, depth characteristics, tracking states (discussed in more detail below), and the like.

The processor of the present invention can be many types of processors and is not limited to any particular type of processor. Additionally, the processor can be multiple processors operating together or independently.

Similarly, the memory of the present invention can be many types of memories and is not limited to any particular type of memory. Additionally, the memory can comprise multiple memories (and multiple types of memories), which can be collocated with each other and/or the processor(s) or remotely located from each other and/or the processor(s).

The memory may comprise logical instructions that, when executed by the processor, cause the processor to generate a third set of data based on the first and/or second sets of data. The third set of data can be generated in real-time. The third set of data may be indicative of estimates of at least one of joint positions and joint angles of the at least a portion of the body over the period of time. In some embodiments, the third data set may be indicative of estimates one or more joint positions of the at least a portion of the body over the period of time. In some embodiments, the third data set may be indicative of estimates one or more joint angles of the at least a portion of the body over the period of time. In some embodiments, the third data set may be indicative of estimates one or more joint positions and joint angles of the at least a portion of the body over the period of time.

In accordance with an exemplary embodiment of the present invention, two Kinect sensors are used, which are referred to as Kinect 1 and Kinect 2. First, data acquired from both Kinects can be transformed into a common coordinate system. This allows the positions collected by each of the markerless sensors to be referenced in the same coordinate system, and thus allows different positions collected by each sensor for the same portion of the object to be detected. Then, the joint position estimates can be combined using sensor fusion, taking into account the tracking state of each joint provided by the Kinects.

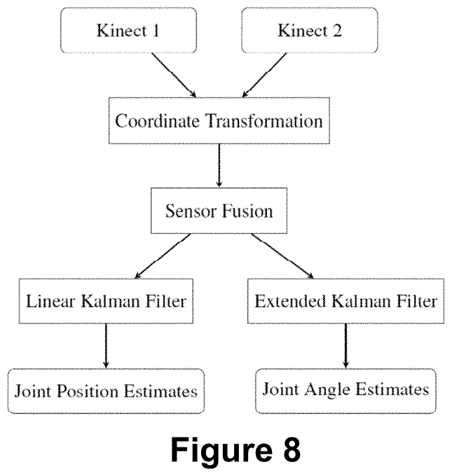

For real-time tracking, the fused data can be subsequently fed into a linear Kalman filter (LKF), yielding joint position estimates based on both Kinect data streams. For offline analysis, the same data is fed into an Extended Kalman filter (EKF). The EKF estimates the joint angles of the upper body model. FIG. 8 shows the workflow of a proposed motion tracking system, in accordance with an exemplary embodiment of the present invention.

Implementation Details

For the real-time portion of the proposed system, the computations are preferably carried out quickly enough to track motion at 30 frames per second. This allows the tracking performance to be perceived without lag. The present invention, however, is not limited to tracking at 30 frames per second. A person skilled in the art would understand that the speed of tracking (e.g., frames per second) can be limited by the speed of the processor and the resolution of the sensors. For example, a sensor with a higher resolution (e.g., collecting positional information on more “pixels”) and/or at greater frame rates would benefit from higher speed processors.

To improve the out-of-the-box skeleton-tracking provided by Kinect, the Dual-Kinect system of the present invention can yield more stable joint position estimates. Compared to a single-Kinect system, using data from two Kinects, as provided by the present invention, can increase the possible tracking volume and reduce problems caused by occlusion, especially for turning motions, e.g., a torso twist.

Hardware and Implementation Restrictions

Development, data collection, and evaluation were carried out on two Laptops with Intel Cores i7-6820HQ CPUs. Because the Kinect for Windows Software Development Kit (SDK) for the second version of Kinect only supports one sensor, data was acquired with two laptops. Communication between the laptops was established via the User Datagram Protocol (UDP), used primarily for low latency applications. In order to directly process the data in MATLAB, the Kin2 Toolbox Interface for MATLAB was used for data collection.

Dual-Kinect Configuration

Embodiments of the present invention may also include methods of positioning markerless sensors. For example, positioning the markerless sensors may comprise positioning the first markerless sensor in a fixed position relative to the body, positioning the second markerless sensor in a temporary position relative to the body, and iteratively altering the position of the second markerless sensor relative to the body by moving the second markerless sensor around the body and checking the accuracy of the estimates of at least one of joint positions and joint angles of the at least a portion of the body over the period of time in the third set of data to determine an optimal position for the second markerless sensor.

Alternatively, positioning the first and second markerless sensors may comprise positioning the first and second markerless sensors adjacent to each other relative to the body, and iteratively altering the position of both the first and second markerless sensors relative to the body by moving both the first and second markerless sensors around the body and checking the accuracy of the estimates of at least one of joint positions and joint angles of the at least a portion of the body over the period of time in the third set of data to determine optimal positions for the first and second markerless sensors.

In any of the methods discussed above, the accuracy may be determined based on a difference between the estimates in the third set of data and estimates determined using a marker-based system, e.g., a Vicon system, or any other type of high-accuracy tracking system. For example, a marker-based system can be considered to provide the “correct” positions of the tracked object. Thus, the “optimal” position for the markerless sensors may be at the positions where the difference between positions identified by a marker-based system and positions identified by the markerless systems is at a minimum (though absolute minimum is not required).

In any of the methods discussed above, the accuracy may be determined based on the tracking states identified by the markerless sensors in the first and second data sets. For example, (as discussed in more detail below), each markerless sensor can provide a tracking state, e.g., for each data point (e.g., pixel), the sensor can indicate whether it sensed an actual specific position, an inferred position, or did not track a position (i.e., no position). Thus, the “optimal” position for the first and second sensors can be the positions for the first and second sensors in which the data sets include the highest number of specific positions sensed or the least number of inferred or no positions sensed.

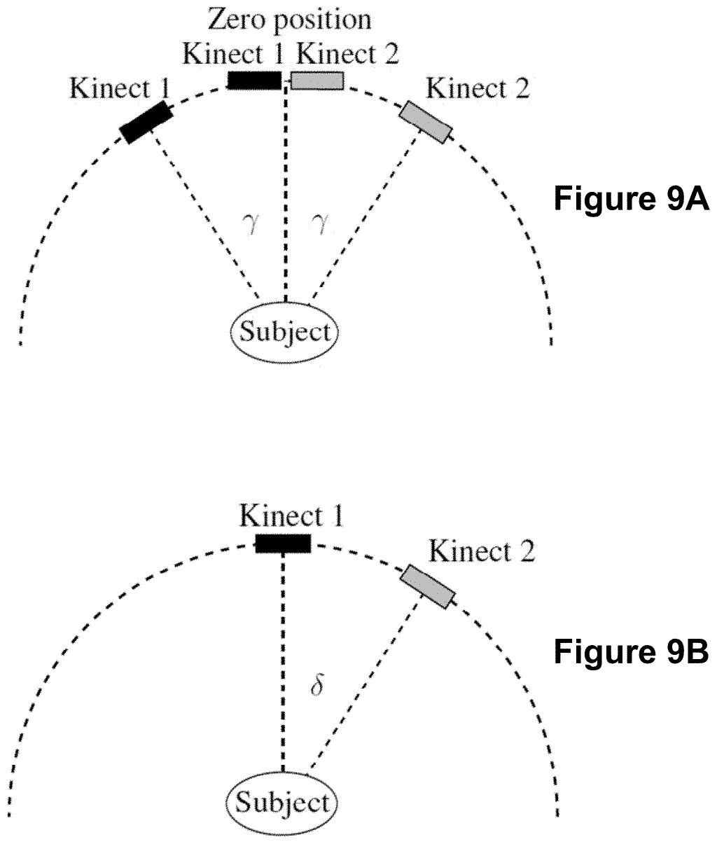

In some embodiments, to find an optimal orientation of the two Kinect sensors relative to each other, and to the test subject, nine different sensor configurations were evaluated. First, both sensors were placed directly next to each other to define the zero position. The test subject stood facing the Kinect sensors at a distance of about two meters, while performing test motions. In accordance with an exemplary embodiment of the present invention, for the first six test configurations, both Kinects were then gradually moved outwards on a circular trajectory around the test subject, as illustrated in FIG. 9A.

The angle γ between each sensor and the zero position was increased in 15° steps as shown in Table 6. In accordance with another exemplary embodiment of the present invention, for configurations 7-9 listed in Table 6, one Kinect sensor was kept at the zero position, while the second Kinect was placed at varying positions on a circular trajectory towards the right of the test subject in 30° steps. The angle δ was measured between the two Kinects, as illustrated in FIG. 9B.

For each sensor configuration, the test subject performed a set of three test motions (a wave motion, a “slow down” signal, and a torso twist). Table 6 lists all tested sensor configurations with their respective angles.

TABLE 6
List of tested Dual-Kinect Configuration Variants

| | Angle between |
|---|---|
| Configuration | Kinect sensors |
| No. | and zero position |
| 1 | γ = 0° |
| 2 | γ = 15° |
| 3 | γ = 30° |
| 4 | γ = 45° |
| 5 | γ = 60° |
| 6 | γ = 75° |
| 7 | δ = 30° |
| 8 | δ = 60° |
| 9 | δ = 90° |

Because the current model is focused on upper body motions, the fused tracking data of the wrist joints was chosen as a measure of tracking quality. Evaluation of the tracking data from the different test configurations showed that with the combined data from both Kinects, the wrist joint could be tracked closely for Configurations 1-5 and Configurations 7-8. However, for Configurations 6 and 9, the wrist trajectory was tracked less reliably, especially at extreme positions during the torso twist motion.

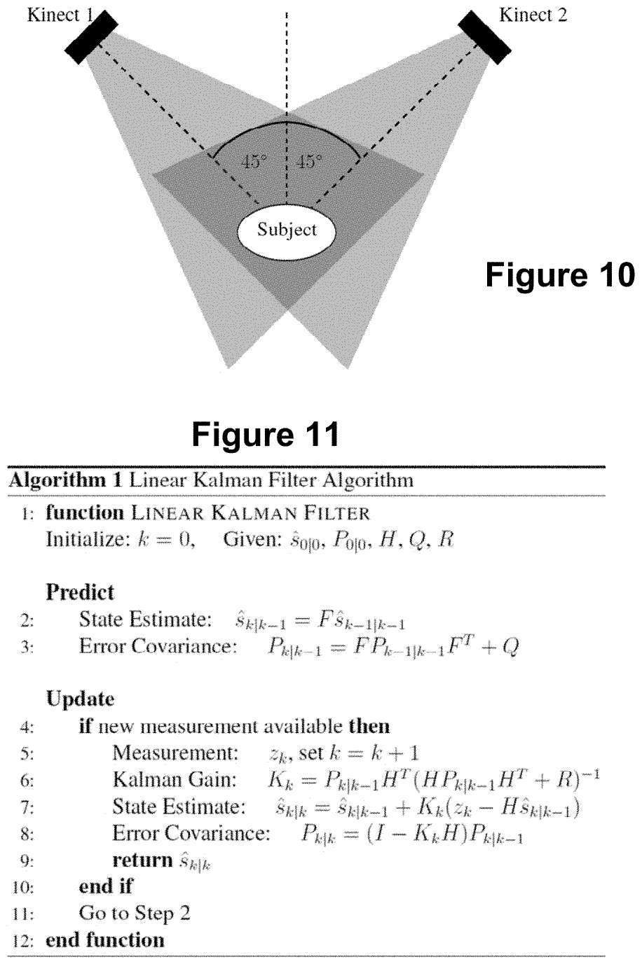

Setting up the Kinects according to Configuration 4, at an angle of 90° with respect to each other, and at an angle of γ=45° to the test subject, produced very good tracking results. The dual-Kinect system was able to cover a large range of motion without losing the wrist position. This configuration was chosen to evaluate the filter performance and comparing the Kinect tracking results to the Vicon motion capture data. The configuration is shown in FIG. 10.

Sensor Calibration and Sensor Fusion

Prior to data collection, the two Kinect sensors were calibrated to yield the rotation matrix and translation vector needed to transform points from the coordinate system of Kinect 2 into a common coordinate system, in this case, the coordinate system of Kinect 1. The present invention, however, does not require that the common coordinate system be the system used with either of the sensors. Rather, the positional information collected by each sensor can be transformed to a common coordinate system different from the system used by the sensors.

Calibration

Considering the need for a fast, real-time calibration without any additional calibration objects, the two Kinects can be calibrated using the initial 3D position estimates of the 25 joints. To ensure no joint occlusion, the test subject stands with straight legs and both arms fully extended, pointing sideways in a T-shape (=T-Pose) for less than two seconds, while 50 frames are acquired by both Kinect sensors. Then, the joint position estimates can be averaged and fed into the calibration algorithm. The coordinate transformation can be calculated via Corresponding Point Set Registration.

Considering two sets of 3D points Set_{A} and Set_{B}, with Set_{A} given in coordinate frame 1 and Set_{B} given in coordinate frame 2, solving for R and t from:

Set_{A}=R·Set_{B}+t Equation 28:

yields the rotation matrix R and translation vector t needed to transform the points from coordinate frame 2 into coordinate frame 1. The process of finding the optimal rigid transformation matrix can be divided into the following steps: (1) find the centroids of both datasets; (2) bring both datasets to the origin; (3) find the optimal rotation R; and (4) find the translation vector t.

The rotation matrix R can be found using Singular Value Decomposition (SVD). Given N Points P_{A} and P_{B} from dataset Set_{A} and Set_{B} respectively, with P=[x y z]^{T}, the centroids of both datasets can be calculated using:

$$\begin{matrix}{centroid}_{A} = \frac{1}{N} {\sum }_{i = 1}^{N} {P}_{A}^{i} & Equation 29 \\ {centroid}_{B} = \frac{1}{N} {\sum }_{i = 1}^{N} {P}_{B}^{i} & Equation 30\end{matrix}$$

The equations needed to find the rotation matrix R are given by:

H=Σᵢ₌₁^{N}(P_{A}ⁱ−centroid_{A})(P_{B}ⁱ−centroid_{B}) Equation 31:

[U,S,V]=SVD(H) Equation 32:

R=V U^{T} Equation 33:

The translation vector t can then be found using:

t=−R*centroid_{B}+centroid_{A} Equation 34:

With the derived rotation matrix and translation vector, the joint position data from Kinect 2 can be transformed into the coordinate system of Kinect 1. Both datasets are further processed in the sensor fusion step to yield fused joint positions.

Sensor Fusion

The present invention can also include a step of fusing the data collected from the two or more sensors, which can allow for a more accurate estimate of positions than using data from only one sensor. As discussed above, the data collected by each sensor can include a tracking position, which, for each data point in the object (e.g., pixel), can include whether the sensor calculated an actual/specific measurement, whether the sensor inferred the measurement, or whether the sensor failed to collect a measurement (i.e., a “no position”). Thus, in some embodiments the fused data can comprise weighted data based on tracking positions with the first and second data sets.

For example, if the first set of data comprises a first specific position for the first portion of the at least a portion of the body at the specific time and the second set of data comprises a second specific position for the first portion of the at least a portion of the body at the specific time, then the third set of data generated by the processor may comprise a weighted position for the first portion of the at least a portion of the body at the specific time, wherein the weighted position is generated using an average of the first and second specific positions. If only one of the first set of data and the second set of data comprises a specific position for the first portion of the at least a portion of the body at the specific time and the other of the first set of data and the second set of data comprises either an inferred position or no position for the first portion of the at least a portion of the body at the specific time, then the third set of data generated by the processor may comprise a weighted position for the first portion of the at least a portion of the body at the specific time, wherein the weighted position is generated using the specific position in the only one of the first set of data and the second set of data but not the inferred position or the no position in the other of the first set of data and the second set of data. If the first set of data comprises a first inferred position for the first portion of the at least a portion of the body at the specific time and the second set of data comprises a second inferred position for the first portion of the at least a portion of the body at the specific time, then the third set of data generated by the processor may comprise a weighted position for the first portion of the at least a portion of the body at the specific time, wherein the weighted position is generated using an average of the first and second inferred positions.

In some exemplary embodiments, the joint positions collected from both Kinects can be used to calculate a weighted fused measurement. In addition to the 3D coordinates of the 25 joints, the Kinect sensor can assign a tracking state to each of the joints, with 0=“Not Tracked,” 1=“Inferred,” and 2=“Tracked.” This information can be used to intelligently fuse the data collected by both Kinects. If the tracking state of a joint is “Tracked” by both Kinects, or the tracking state of the joint is “Inferred” in both Kinects, then the average position is taken. If a joint is “Tracked” by one Kinect, but “Infrerred” or “Not Tracked” by the other, then the fused position only uses data from the “Tracked” joint. The fused position p_{fused} of each joint can, therefore, be calculated using the position estimates p₁ from Kinect 1 and p₂ from Kinect 2 as follows:

p_{fused}=w₁p₁+w₂p₂, Equation 35:

with weighting factors w₁ and w₂ assigned using the tracking state information for each joint obtained from both Kinects:

$$\begin{matrix}{w}_{1} = \frac{TrackingState 1}{TrackingState 1 + TrackingState 2} & Equation 36 \\ {w}_{2} = \frac{TrackingState 2}{TrackingState 1 + TrackingState 2} & Equation 37\end{matrix}$$

Linear Kalman Filter for Kinect Joint Tracking

To improve tracking of the 25 joints, two versions of a linear Kalman filter were designed based on the state space models discussed above. The state vector can be taken to be the true 3D coordinates of the 25 joints for the zero-velocity model, and the 3D coordinates and velocities of the 25 joints for the constant-velocity model. For the sake of simplicity, the derived Kalman filter equations are presented for only one joint, but the same equations can be applied to any number of tracked joints.

Linear Kalman Filter Implementation

After completing the coordinate transformation and sensor fusion steps described above, the fused joint position can be fed into the Kalman filter as a measurement. Algorithm 1, which is shown in FIG. 11, summarizes the linear Kalman filter algorithm used for the joint position tracking with the Dual-Kinect system, in accordance with an exemplary embodiment of the present invention.

The filter equations can remain the same for both the zero and the constant-velocity model.

Depending on the chosen underlying state space model, the state vector, as well as state transition matrix F and the observation matrix H are set accordingly. For the zero-velocity model, the state vector includes the joint positions s=[x y z]^{T}, and the matrices take the following form:

$$F = [ \begin{matrix}1 & 0 & 0 \\ 0 & 1 & 0 \\ 0 & 0 & 1\end{matrix} ] , H = [ \begin{matrix}1 & 0 & 0 \\ 0 & 1 & 0 \\ 0 & 0 & 1\end{matrix} ] \qquad Equation 38$$

For the constant-velocity model, the states are the joint positions and the joint velocities s=[x y z \dot{x} \dot{y} ż]^{T}, and F and H are calculated as follows:

$$F = [ \begin{matrix}1 & 0 & 0 & Δ t & 0 & 0 \\ 0 & 1 & 0 & 0 & Δ t & 0 \\ 0 & 0 & 1 & 0 & 0 & Δ t \\ 0 & 0 & 0 & 1 & 0 & 0 \\ 0 & 0 & 0 & 0 & 1 & 0 \\ 0 & 0 & 0 & 0 & 0 & 1\end{matrix} ] , H = [ \begin{matrix}1 & 0 & 0 & 0 & 0 & 0 \\ 0 & 1 & 0 & 0 & 0 & 0 \\ 0 & 0 & 1 & 0 & 0 & 0\end{matrix} ] \qquad Equation 39$$

In both cases, the measurements can be the fused joint positions from the Dual-Kinect system.

Extended Kalman Filter for Kinect Joint Tracking

In accordance with an exemplary embodiment of the present invention, to implement the extended Kalman filter, nonlinear dynamics of upper body motions can be taken into account. The joint positions can be calculated using the transformation matrices derived from the kinematic human upper body model discussed above. Instead of the joint position and translational joint velocities used with the linear Kalman filter, the joint angles and angular joint velocities can be taken to be the states of the system: s=[θ₁ . . . θ₂₆ \dot{θ}₁ . . . \dot{θ}₂₆]^{T}

Assuming constant angular joint velocities, the system can have the following description in sampled time:

sₖ₊₁=f(sₖ)+wₖ=Fsₖ+wₖ Equation 40:

zₖ=h(sₖ)+vₖ Equation 41:

The process noise wk and the measurement noise vₖ can be assumed to be zero mean, Gaussian noise with covariance Qₖ and Rₖ, respectively. The state transition matrix can be given by:

$$F = [ \begin{matrix}{26 \times 26}_{} & Δ t {26 \times 26}_{} \\ {0}_{26 \times 26} & {26 \times 26}_{}\end{matrix} ] \qquad Equation 42$$

with sampling time Δt. In the measurement model, the 3D positions of the upper body joints can be calculated using the DH-Parameters and transformation matrices for the upper body model discussed above. Recalling the transformation matrices:

$${T}_{i}^{i + 1} = [ \begin{matrix}cos {θ}_{i} & - sin {θ}_{i} cos {α}_{i} & sin {θ}_{i} sin {α}_{i} & {a}_{i} cos {θ}_{i} \\ sin {θ}_{i} & cos {θ}_{i} cos {α}_{i} & - cos {θ}_{i} sin {α}_{i} & {a}_{i} sin {θ}_{i} \\ 0 & sin {α}_{i} & cos {α}_{i} & {d}_{i} \\ 0 & 6 & 0 & 1\end{matrix} ] \qquad Equation 43$$

the spatial configuration of the upper body model is defined for given link lengths L₁, . . . , L₁₀ and joint angles θ₁, . . . , θ₂₆. Using the transformation matrices, Tⁱᵢ₋₁=Tᵢ₋₁ⁱ(θᵢ), the position of the i^{th} joint pᵢ=[xᵢ yᵢ zᵢ]^{T} can be expressed as a function of i joint angles:

$$\begin{matrix}[ \begin{matrix}{p}_{i} \\ 1\end{matrix} ] = {T}_{0}^{1} {T}_{1}^{2} … {T}_{i - 1}^{i} [ \begin{matrix}0 \\ 0 \\ 0 \\ 1\end{matrix} ] & Equation 44 \\ = h ( {θ}_{1} , ⋯ , {θ}_{i} ) = h ( s ) & Equation 45\end{matrix}$$

The system can be linearized about the current state estimate using the Jacobian:

$${H}_{k} = \frac{\partial h}{\partial s} {\vert}_{s = {\hat{s}}_{k}} \qquad Equation 46$$

For each time step k, the linearized function can be evaluated at the current state estimate. The form of the underlying transformation matrices Tᵢ₋₁ⁱ can be dependent on the body segment lengths L₁-L₁₀. Therefore, h(s) can be initialized with corresponding values for the body segment lengths of each individual test subject obtained during the Dual-Kinect calibration process.

Extended Kalman Filter Implementation

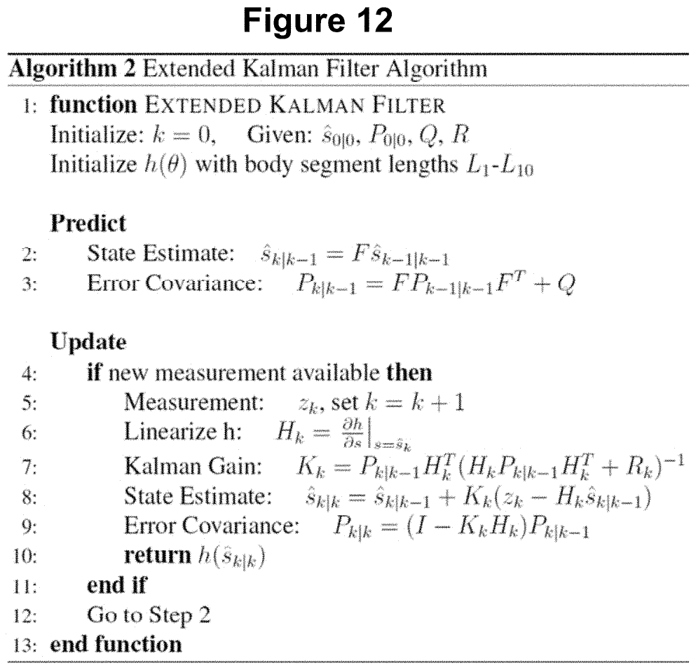

Algorithm 2, which is shown in FIG. 12, summarizes the extended Kalman filter algorithm used for upper body joint tracking, in accordance with an exemplary embodiment of the present invention.

Handling Missing Data

One advantage of the underlying state space model for the Kalman filter is that a missing observation can easily be integrated into the filter framework. If at time step k a joint's position is lost by both Kinect sensors (tracking state “Not Tracked” for Kinect 1 and Kinect 2), then the vector zₖ−Hₖŝ_{k|k-1} and the Kalman gain Kₖ are set to zero. Thus, the update can follow the state space model:

ŝ_{k|k}=Fŝ_{k-1|k-1} Equation 47:

P_{k|k}=FP_{k-1|k-1}F^{T}+Q Equation 48:

This approach can be applied to the implementations of both the linear Kalman filter and the extended Kalman filter.

Experimental Setup

Tracked Motions: Joint tracking with an inventive Dual-Kinect system utilizing the Kalman filters was tested with three test motions: a two-handed wave, a two-handed “slow down” signal, and a torso twist. The torso twist motion was helpful to determine the effect of joint occlusion on the Dual-Kinect system. The test subject rotated her upper body from side to side about 90 degrees, which causes joint occlusion of the elbow, wrist, and hand. Starting from the T-Pose, the test subject performed five repetitions of all three test motions. To clearly distinguish the between different motions in the recorded data, the subject returned to the T-Pose for about two seconds before switching to a new motion. Data was recorded continuously until five repetitions for each of the three motions had been completed, and the subject had returned to the T-Pose.

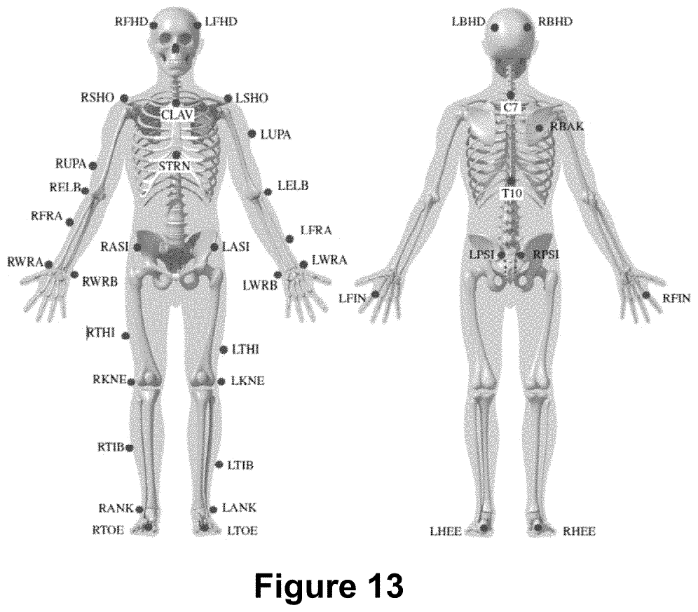

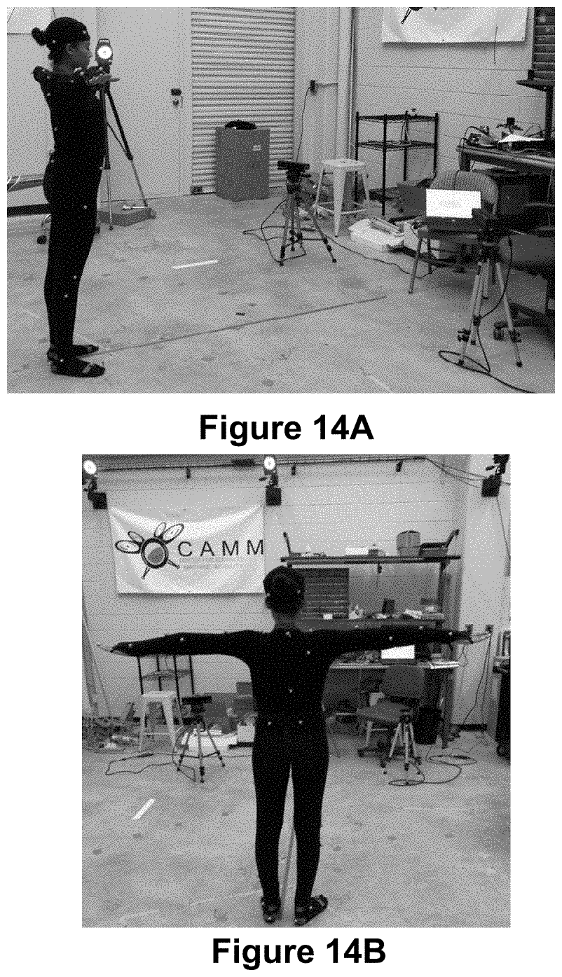

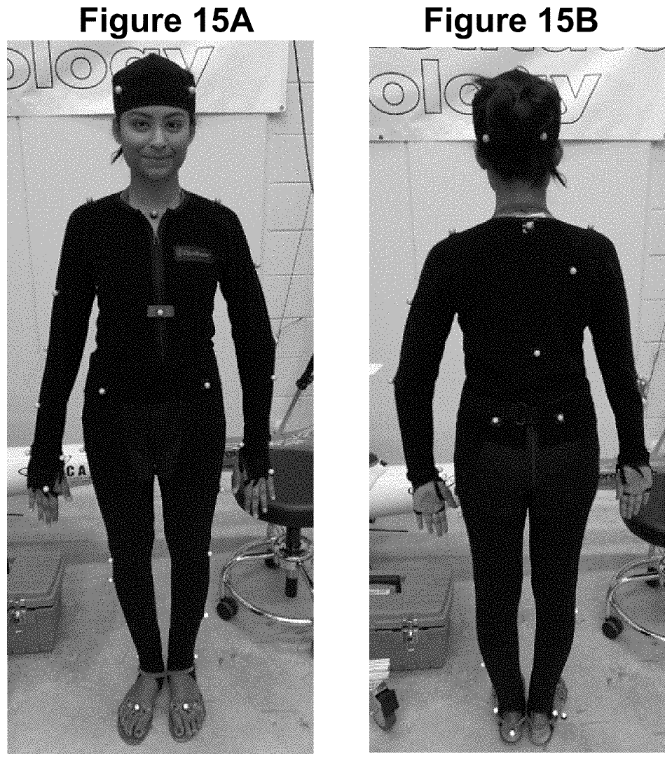

Marker-based Tracking: To evaluate the performance of the Dual-Kinect system, tracking data for the three test motions was compared to marker-based tracking data recorded with a Vicon 3D motion capture system at the Indoor Flight Facility at Georgia Tech. For the marker-based motion capture with the Vicon system, the full body Plug-in-Gait marker setup was used. The marker setup uses 39 retroreflective markers and can be used with the Plug-in-Gait model, which is a well-established, and commonly-used, model for marker-based motion capture. FIG. 13 shows the locations of the markers for the full body Plug-in-Gait model. FIGS. 14A-B show the subject standing in the T-Pose while facing the Dual-Kinect setup. FIGS. 15A-B show the test subject wearing the motion capture suit with the attached markers.

Marker Trajectory Data Processing: Motion capture data from the Vicon system was processed in the Vicon Nexus 2.5 and Vicon BodyBuilder 3.6.3 software (Vicon Motion Systems, Oxford, UK). Marker trajectories were filtered using a Woltring filter. Gaps in the marker data with durations <20 frames (<0.2 seconds) were filled using spline interpolation. To compare the performance of the inventive Dual-Kinect system to the marker-based Vicon tracking, joint center locations corresponding to the joints tracked by the Kinect system were calculated from the marker trajectories in Vicon BodyBuilder.

Results and Comparison with Vicon Motion Capture

In this section, results from tracking experiments with two variants of the linear Kalman filter and the Extended Kalman filter (EKF) are presented. While the first variant of the linear Kalman filter (LKF1) uses a zero-velocity model, the second variant (LKF2) uses a constant-velocity motion model. The position estimates are compared to the raw data from the Kinect sensor, and to joint position data obtained from marker-based motion capture. The joint positions derived from the Vicon system were assumed to be the true positions of the joints.

Linear Kalman Filter

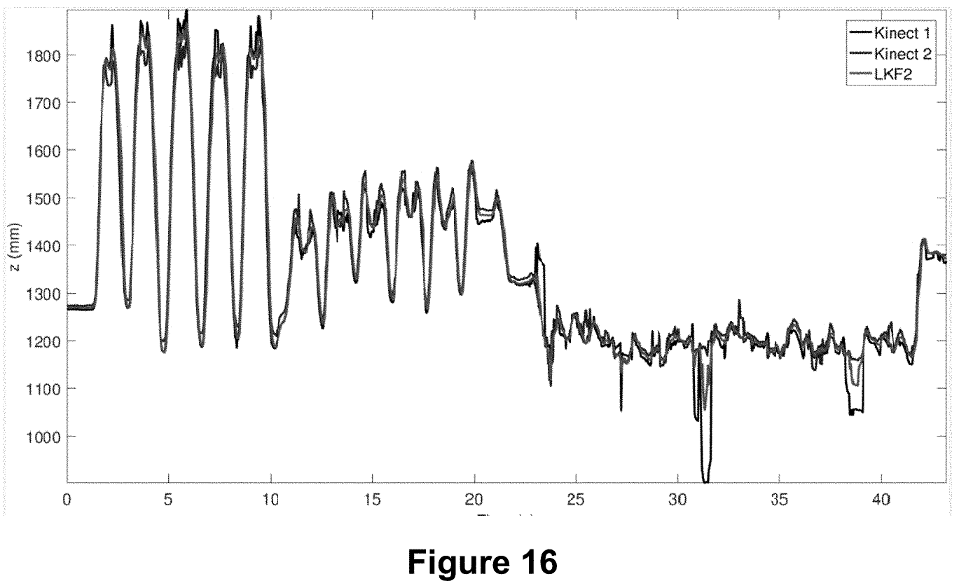

During the experiments, it was noted that the differences between the two variants of the linear Kalman filter were in many cases small, but became larger as the process covariance was decreased. This result is to be expected, as a smaller process covariance means the filter relies more on the underlying motion model and less on actual observations. FIG. 16 shows the z component of the left wrist joint position for the recorded test motions estimated with the linear Kalman filter using the constant-velocity model (LKF2). The position estimate is compared with the raw data acquired by Kinects 1 and 2.

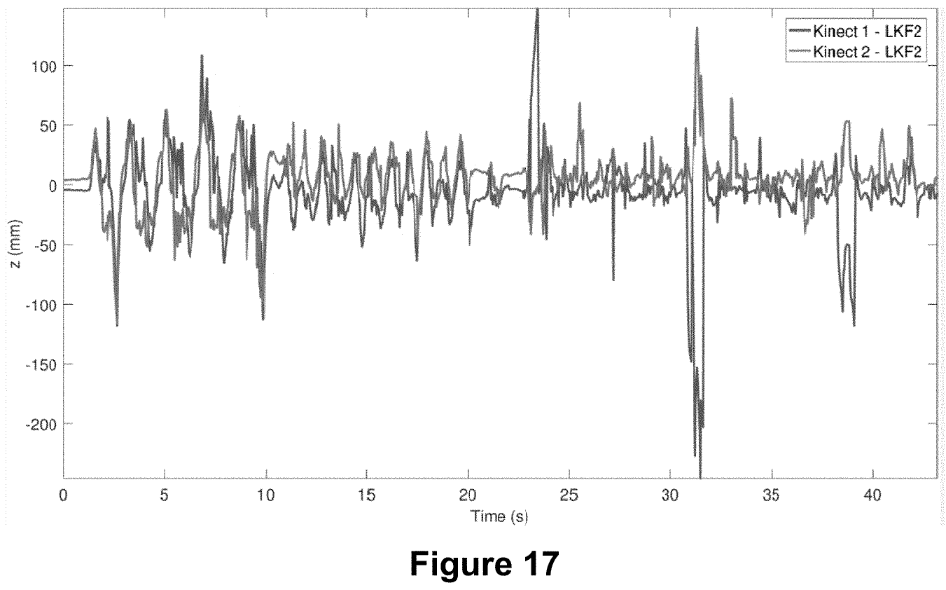

FIG. 17 shows the difference between the raw data and the filtered data for the z component of the left wrist position estimate. The greatest deviation between the raw data and the LKF2 output was observed during the torso twist motion, as the wrist moved behind the torso during the motion, and was therefore occluded. The average deviation between the Kinect 1 and the LKF2 output was 19.6113 mm, and the maximum deviation between Kinect 1 and the LKF2 output was 246.0466 mm. The average deviation between the Kinect 2 and LKF2 was 16.3035 mm and the maximum deviation between Kinect 2 and LKF2 was 131.5598 mm.

To compare the joint tracking data from Kinect with Vicon data, the filter outputs were aligned with the Vicon data in terms of motion timing and were transformed into the Vicon's coordinate system. Because the Kinect samples at a rate of approximately 30 Hz, the filter outputs were interpolated using linear interpolation to match the Vicon's sampling rate of 100 Hz.

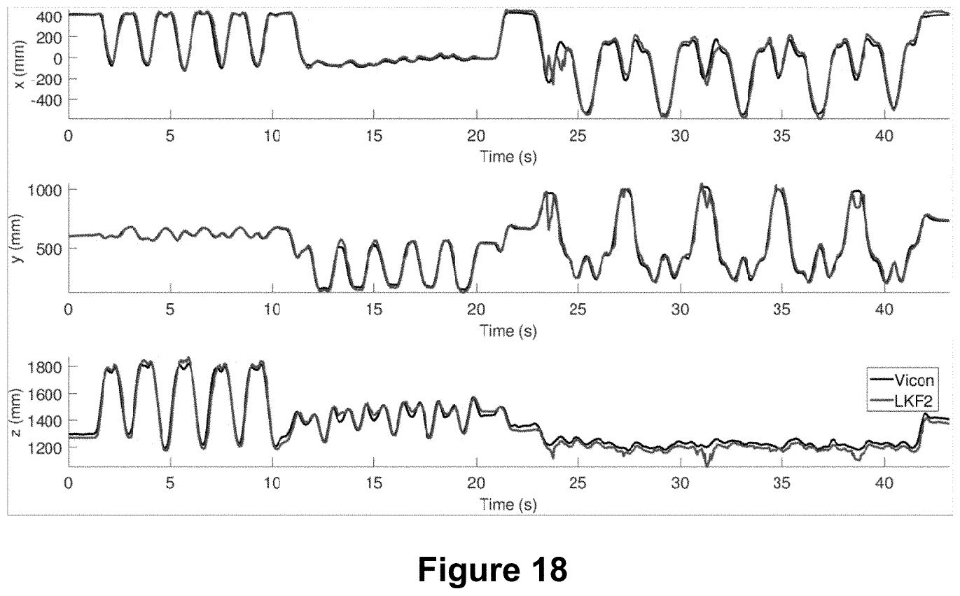

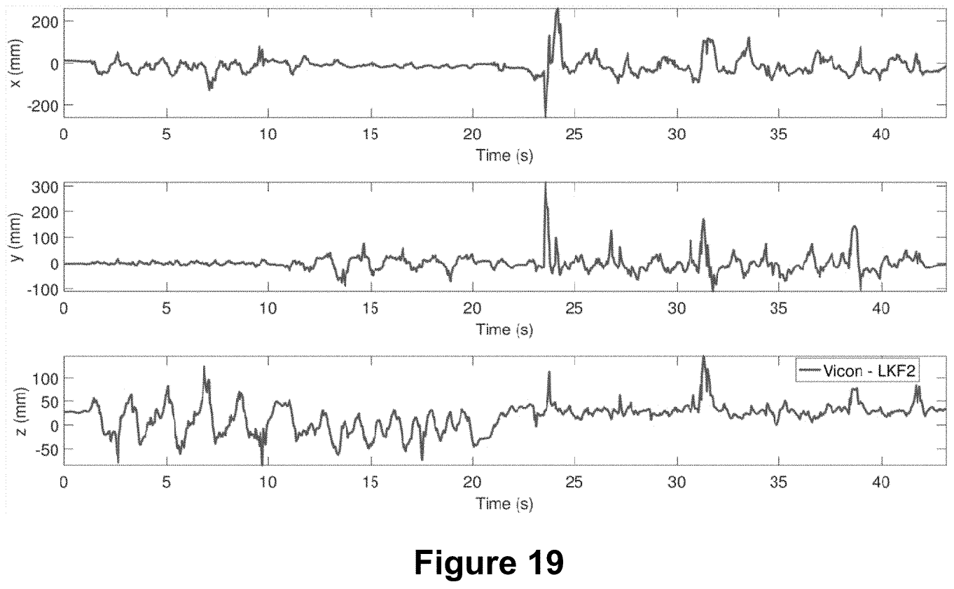

FIG. 18 shows the position estimate of the left wrist from the LKF2. The results are compared to the joint trajectory obtained with the Vicon system. FIG. 19 shows the difference between the Vicon and the LKF2 data for tracking the left wrist position. The mean and maximum deviations between the LKF2 output and the Vicon data are listed in Table 7. The mean deviation was smallest in the y component of the position estimate, and was worst in the x direction. The maximum deviation also occurred in the x direction.

TABLE 7
Mean Absolute Error (MAE) and Maximum Absolute
Error (MAD) for LKF2 for Tracking the
Position of the Left Wrist. All values in min.

| | x | y | z |
|---|---|---|---|
| MAF | 16.9908 | 16.1585 | 22.9192 |
| MAD | 163.7479 | 157.4331 | 125.5755 |

FIG. 18 shows that the left wrist position was closely tracked for the wave motion (from t=0 s until t=10 s) and the “slow down” motion (from t=11s until 1=21 s). During the torso twist motion starting at t=23 s, however, there was some discrepancy between Kinect and Vicon tracking data for extreme positions, when the wrist moved out of the field of view of both Kinect sensors. Generally, the wrist could be tracked well for the majority of the test motions.

Extended Kalman Filter

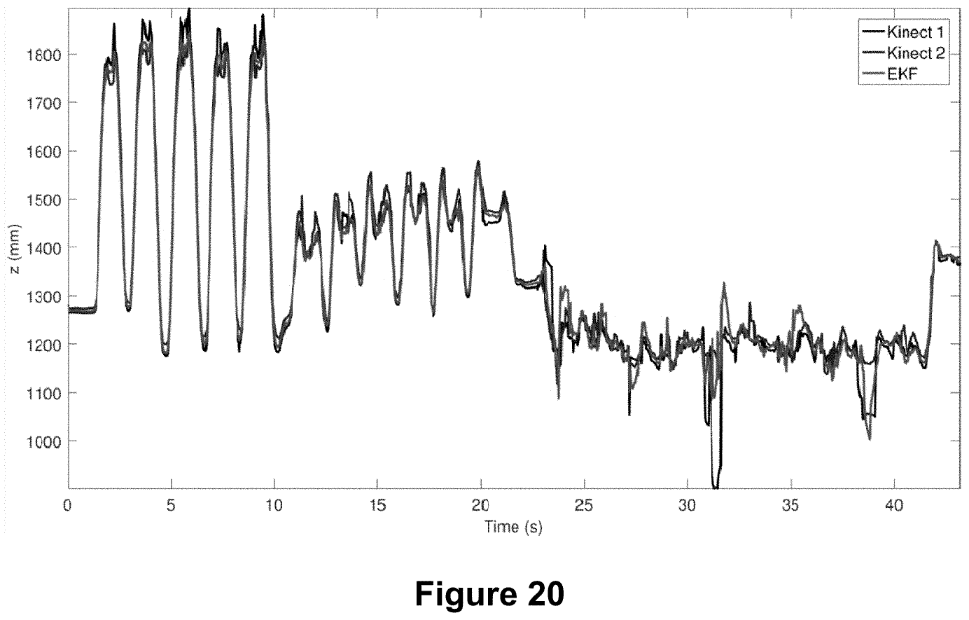

FIG. 20 presents the z component of the left wrist joint trajectory from the EKF output, as well as the raw data acquired by Kinect 1 and 2. The wrist position could be tracked closely for the first two motions (two-handed wave and “slow down signal”). However, the EKF outputs from tracking the torso twist motion were not as smooth as the linear Kalman filter outputs. To better compare the tracking performance of the different filter variants, the same data sets obtained from Kinect 1 and 2 were used.

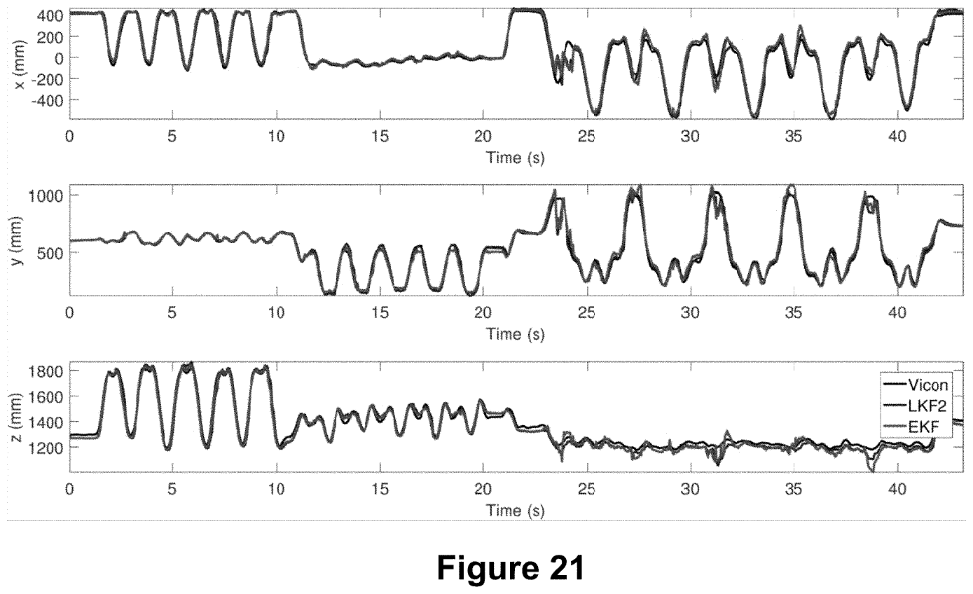

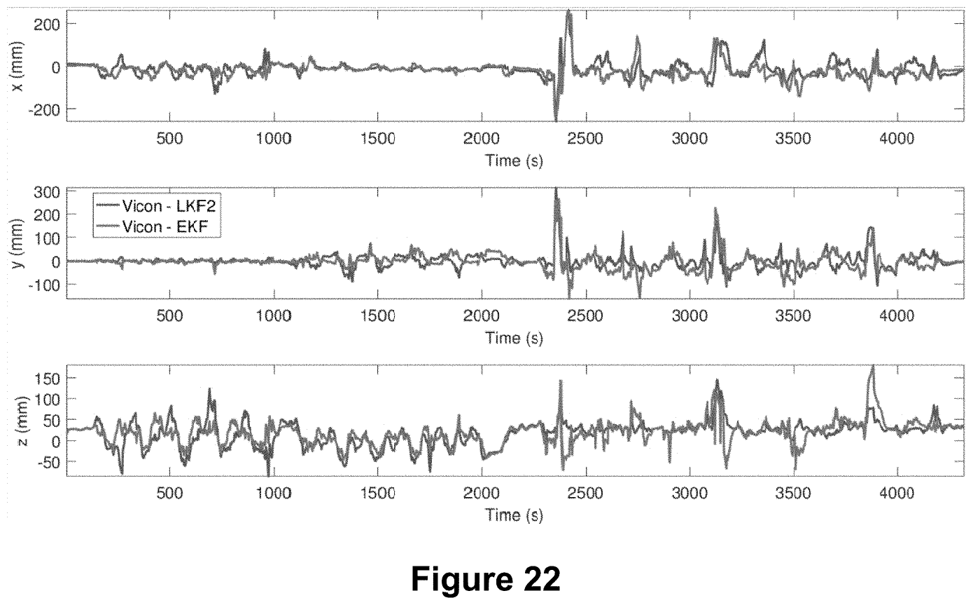

FIG. 21 compares the wrist position estimate from the EKF with the LKF2 outputs and the data obtained with the Vicon system. FIG. 22 shows the deviation between each filter output and the Vicon data. For the first two tracked motions, differences between the filter outputs are very small. For the torso twist motion, the linear Kalman filter provides a more stable and smoother tracking of the joint position.

To evaluate accuracy of the tracking with the different variants of the Kalman filters, the mean absolute errors in x, y, and z position between the filter outputs and joint position data collected with the Vicon system were calculated for ten joints considered in the kinematic upper body model discussed above: SpineMid, SpineShoulder, ShoulderLeft, ElbowLeft, WristLeft, HandTipLeft, ShoulderRight, ElbowRight, WristRight, and HandTipRight.

Table 8 lists the mean absolute error in x, y, and z position averaged over the ten joints considered in the upper body model. In general, the different filter variants tracked the motion of the joints with similar accuracy, with the linear Kalman filter using a zero-velocity model (LKF1) performing slightly better than the linear Kalman filter using a constant-velocity model (LKF2) and the Extended Kalman filter (EKF). The most accurate results in terms of least mean absolute error averaged over all joints were achieved while tracking the z coordinate of the position (along the vertical axis). In general, mean absolute error was greatest in the y direction (corresponds to the axes extending from the Kinect sensors to the test subject).

TABLE 8
Mean Absolute Error (MAE) for all Filter Variants
Averaged Over Ten Upper Body Joints

| Filter | MAE (mm) | | |
|---|---|---|---|
| variant | x | y | z |
| LKF 1 | 36.4419 | 37.0706 | 30.4291 |
| LKF 2 | 36.5739 | 36.9309 | 31.0161 |
| EKF | 37.5273 | 40.2169 | 32.3851 |

The Kinect's out-of-the-box joint tracking algorithm is not based on a kinematic model for the human body. As a consequence, the distances between neighboring tracked joints, i.e. the limb lengths of the estimated skeleton are not kept constant. This can lead to unrealistic variation of the body segment lengths and “jumping” of the joint positions. The extended Kalman filter used in this embodiment of the invention uses the novel kinematic human upper body model discussed above. By using the model, constant limb lengths are enforced during the joint tracking.

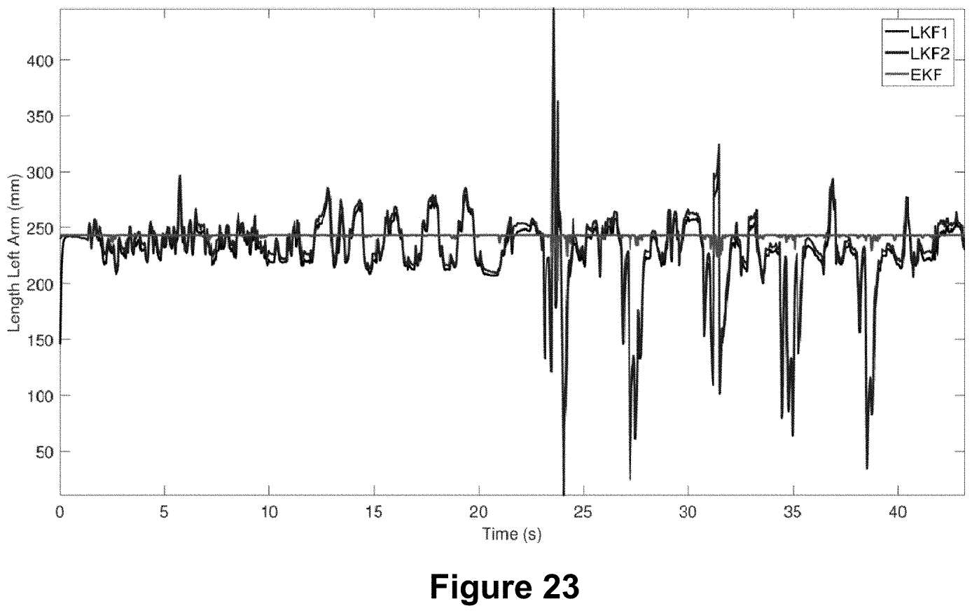

FIG. 23 shows the length of the left arm calculated from the different filter outputs. The arm length was measured from elbow joint to wrist joint. The outputs from the EKF show that by definition, the arm length was kept constant throughout the motion, while the estimates from the linear Kalman filters show that the estimated arm length varied over time.

Tracking with Garments of Different Fit

Experiments were also conducted to determine how the fit of clothing affects motion capture and joint tracking with an inventive dual-Kinect system. Most motion capture systems require extremely tight fitting clothes, very little clothing, or a special suit to track joint position and angles accurately. Moreover, a large number of these systems are marker-based systems that use retroreflective markers to track joints. In the event that the test subject wears glasses, light colored clothing, or reflective jewelry, the data becomes noisy. Given that the Kinect sensor uses RGB and depth data to track a human-shaped silhouette, it benefits with a reasonable view of the joint motions that compose the human body motion. Clothing worn by the test subject obscures the visible joint motion to some degree. These experiments demonstrate that the inventive dual-Kinect system can track human motion even when relatively loose clothing is worn by the test subject.

The Kinects were placed according in Configuration 4 (discussed above), at an angle of 90° with respect to each other, and at an angle of γ=45° to the test subject. The test subject executed characteristic motion performed by people to test fit of garments, such as the torso twist, calf extensions, and squats. Joint position data was collected for two trials, one with fitted clothing, and the other with loose clothing. The skeleton tracked by the dual-Kinect system is overlaid on the RGB frame of a video recording of the test motions.

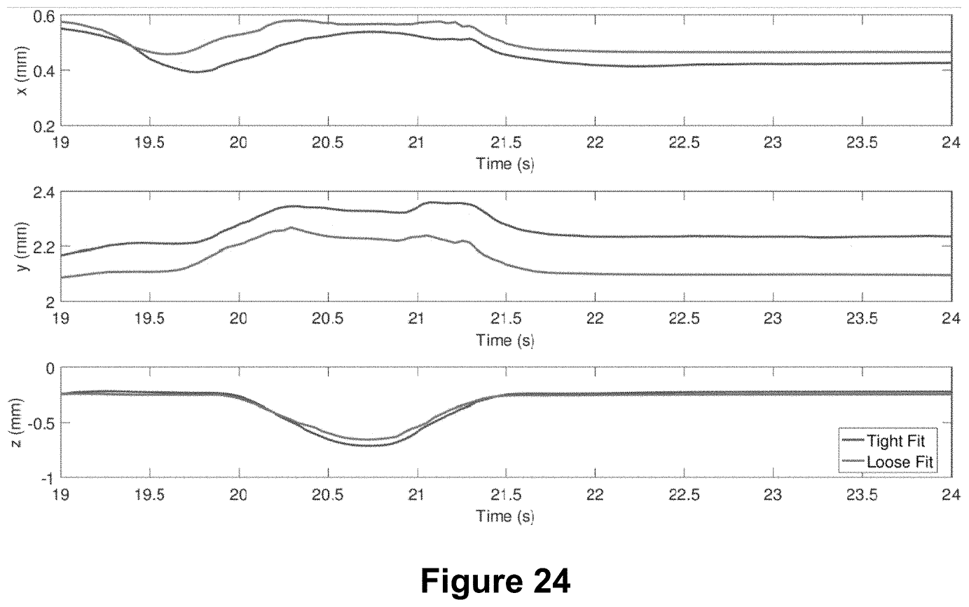

FIG. 24 shows the joint position plot for the SpineBase from the two trials. The subject performed two calf extensions and a squat. In the z component of the tracked joint, the squat motion can be clearly identified starting from t=20 s until t=22.5 s for tracking with both tight-fitting and loose-fitting clothes. Because the test subject changed starting positions in between the two trials, there was an offset in the x and y component of the tracked position. It could be observed that loose fitting clothing did not significantly degrade the tracking ability of the dual-Kinect system. Because the tracking does not fail with the loose fit of the clothing, it can be concluded that, in general, the dual-Kinect system is a robust tool to capture motions performed by clothed test subjects.

Graphical User Interface for Real-Time Joint Tracking with Dual-Kinect

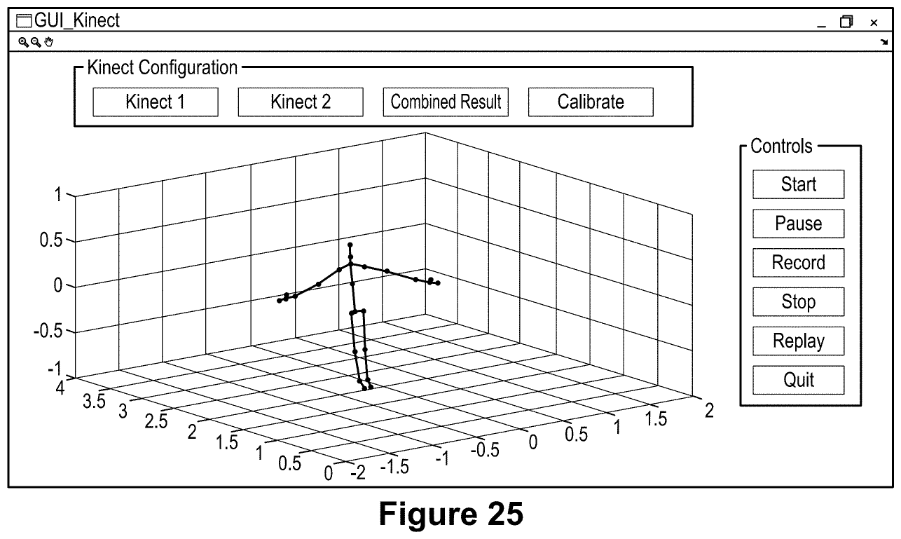

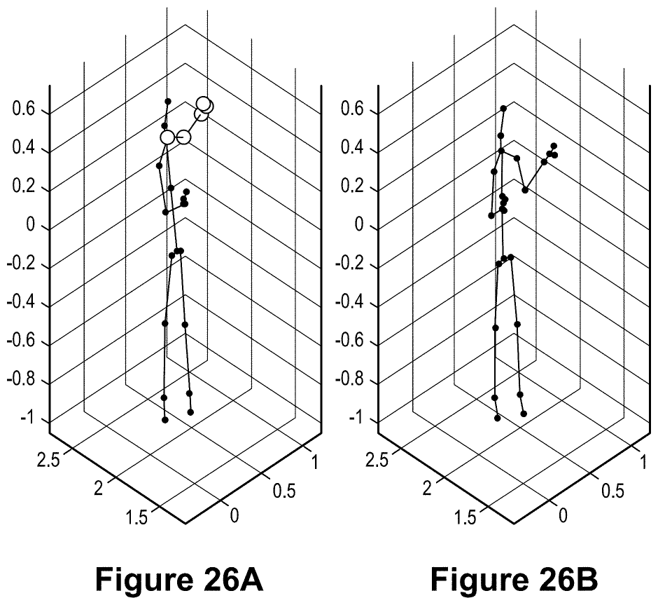

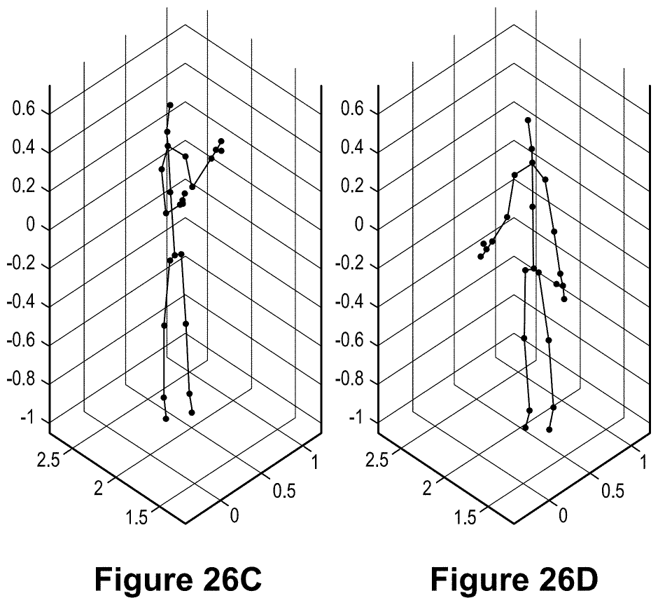

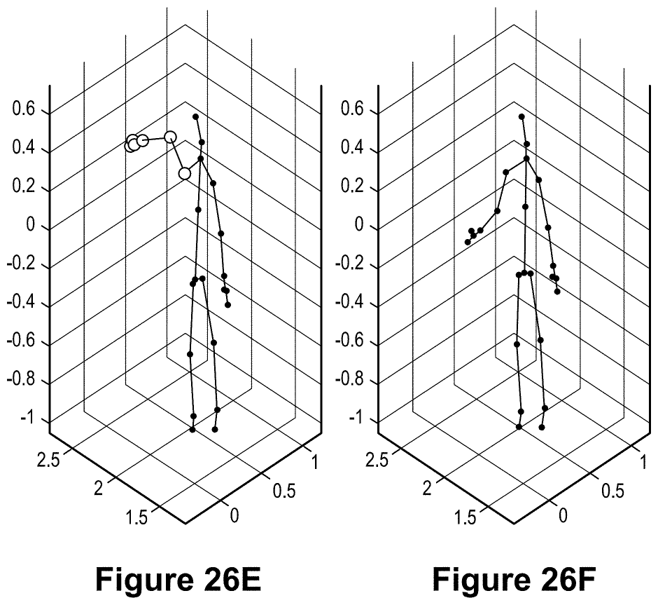

To visualize the real-time tracking with the Dual-Kinect system, a graphical user interface (GUI) was implemented in MATLAB. FIG. 25 shows the implemented GUI. FIGS. 26A-F show example results for tracking the test motions ((a)-(c) torso twist, (d)-(f) two-handed wave motion). The tracked skeletons from both Kinect sensors, as well as the combined resulting skeleton are plotted for each time frame. The GUI can be used for calibration, recording tracking data, and replaying the tracked results.

A red colored joint indicates that the Kinect sensor has either lost the joint's position completely, or the tracking state of the joint is ‘Inferred’. As shown in FIGS. 26A-F, the fused data compensates for occlusion of the joints of the right arm and uses the more realistic position data from Kinect 2 to calculate the position estimation.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the disclosed technology is defined by the claims appended hereto.

1. A system comprising:
a first markerless sensor configured to generate a first set of data indicative of positions of at least a portion of a body over a period of time;
a second markerless sensor configured to generate a second set of data indicative of positions of the at least a portion of the body over the period of time;
a processor; and
a memory comprising logical instructions that, when executed by the processor, cause the processor to process the first and second sets of data using an extended Kalman filter.

2. The system of claim 1, wherein the memory further comprises instructions that, when executed by the processor, cause the processor to generate a third set of data based on the first and second sets of data.

3. (canceled)

4. The system of claim 2, wherein the third set of data is indicative of joint positions of the at least a portion of the body over the period of time.

5. (canceled)

6. The system of claim 2, wherein the third set of data is indicative of joint angles of the at least a portion of the body over the period of time.

7. The system of claim 1, wherein the first set of data includes data points indicative of a position for a plurality of predetermined portions of the at least a portion of the body over the period of time, and wherein the second set of data includes data points indicative of a position for the plurality of predetermined portions of the at least a portion of the body over the period of time.

8. The system of claim 7, wherein for each of the plurality of predetermined portions of the at least a portion of the body, the first and second sets of data indicate either a specific position for that portion of the at least a portion of the body, an inferred position for that portion of the at least a portion of the body, or no position for that portion of the at least a portion of the body.

9. A system comprising:
a first markerless sensor configured to generate a first set of data indicative of positions of at least a portion of a body over a period of time, wherein at least a portion of the first set of data indicates one or more of:
a specific position of a first portion of the body;
an inferred position of the first portion of the body; and
no position of the first portion of the body;
a second markerless sensor configured to generate a second set of data indicative of positions of the at least a portion of the body over the period of time, wherein at least a portion of the second set of data indicates one or more of:
a specific position of the first portion of the body;
an inferred position of the first portion of the body; and
no position of the first portion of the body;
a processor; and
a memory comprising logical instructions that, when executed by the processor, cause the processor to generate a third set of data based on at least a portion of the first and second sets of data;
wherein if the first set of data comprises a first specific position for the first portion of the body at a specific time and the second set of data comprises a second specific position for the first portion of the body at the specific time, then the third set of data comprises a weighted position for the first portion of the body at the specific time, wherein the weighted position is generated using an average of the first and second specific positions.

10. A system comprising:
a first markerless sensor configured to generate a first set of data indicative of positions of at least a portion of a body over a period of time, wherein at least a portion of the first set of data indicates one or more of:
a specific position of a first portion of the body;
an inferred position of the first portion of the body; and
no position of the first portion of the body;
a second markerless sensor configured to generate a second set of data indicative of positions of the at least a portion of the body over the period of time, wherein at least a portion of the second set of data indicates one or more of:
a specific position of the first portion of the body;
an inferred position of the first portion of the body; and
no position of the first portion of the body;
a processor; and
a memory comprising logical instructions that, when executed by the processor, cause the processor to generate a third set of data based on at least a portion of the first and second sets of data;
wherein if only one of the first set of data and the second set of data comprises a specific position for the first portion of the body at a specific time and the other of the first set of data and the second set of data comprises either an inferred position or no position for the first portion of the body at the specific time, then the third set of data comprises a weighted position for the first portion of the body at the specific time, wherein the weighted position is generated using the specific position but not the inferred position or no position.

11. A system 8 comprising:
a first markerless sensor configured to generate a first set of data indicative of positions of at least a portion of a body over a period of time, wherein at least a portion of the first set of data indicates one or more of:
a specific position of a first portion of the body;
an inferred position of the first portion of the body; and
no position of the first portion of the body;
a second markerless sensor configured to generate a second set of data indicative of positions of the at least a portion of the body over the period of time, wherein at least a portion of the second set of data indicates one or more of:
a specific position of the first portion of the body;
an inferred position of the first portion of the body; and
no position of the first portion of the body;
a processor; and
a memory comprising logical instructions that, when executed by the processor, cause the processor to generate a third set of data based on at least a portion of the first and second sets of data;
wherein if the first set of data comprises a first inferred position for the first portion of the body at a specific time and the second set of data comprises a second inferred position for the first portion of the body at the specific time, then the third set of data comprises a weighted position for the first portion of the body at the specific time, wherein the weighted position is generated using a weighted average of the first and second inferred positions.

12. The system of claim 7, wherein the plurality of predetermined portions of the at least a portion of the body comprise one or more joints in at least a portion of a human body.

13. The system of claim 1, wherein the at least a portion of a body comprises the upper body of a human.

14. The system of claim 1, wherein the at least a portion of a body comprises the lower body of a human.

15. The system of claim 1, wherein the memory further comprises instructions that, when executed by the processor, cause the processor to transform the positions in at least one of the first set of data and the second set of data into a common coordinate system.

16. A method comprising:
generating a first set of data with a first markerless sensor, the first set of data indicative of positions of a portion of a body over a specific period of time, wherein at least a portion of the first set of data indicates one or more of:
a specific position of a first portion of the body;
an inferred position of the first portion of the body; and
no position of the first portion of the body;
generating a second set of data with a second markerless sensor, the second set of data indicative of positions of the portion of the body over the specific period of time, wherein at least a portion of the second set of data indicates one or more of:
a specific position of the first portion of the body;
an inferred position of the first portion of the body; and
no position of the first portion of the body; and
processing at least a portion of the first and second sets of data to generate a third set of data, the third set of data including a weighted position for the first portion of the body at the specific time;
wherein the weighted position is generated using one of:
an average of a first and second specific positions if the first set of data comprises the first specific position for the first portion of the body at the specific time and the second set of data comprises the second specific position for the first portion of the body at the specific time;
a specific position but not an inferred position or no position if only one of the first set of data and the second set of data comprises the specific position for the first portion of the body at the specific time and the other of the first set of data and the second set of data comprises either the inferred position or no position for the first portion of the body at the specific time; and
a weighted average of a first and second inferred positions if the first set of data comprises the first inferred position for the first portion of the body at the specific time and the second set of data comprises the second inferred position for the first portion of the body at the specific time.

17. The method of claim 16 further comprising transforming positions in at least one of the first and second sets of data into a common coordinate system.

18. The method of claim 16, wherein the first set of data includes data points indicative of a position for a plurality of predetermined portions of the portion of the body over the specific period of time; and
wherein the second set of data includes data points indicative of a position for the plurality of predetermined portions of the portion of the body over the specific period of time.

19. The method of claim 18, wherein the plurality of predetermined portions of the portion of the body comprise one or more joints in at least a portion of a human body.

20. The method of claim 18 further comprising fusing the first and second sets of data to generate a fourth set of data indicative of weighted positions of the portion of the body over the specific period of time, the weighted positions based off of the positions in the first set of data, positions in the second set of data, or a combination thereof.

21.-24. (canceled)

25. The method of claim 20 further comprising processing the fourth set of data with a Kalman filter.

26. The method of claim 25, wherein the Kalman filter is a linear Kalman filter.

27. The method of claim 26, wherein processing the fused positions with the linear Kalman filter generates data indicative of joint positions of the portion of the body over the specific period of time.

28. The method of claim 25, wherein the Kalman filter is an extended Kalman filter.

29. The method of claim 28, wherein processing the fused positions with the extended Kalman filter generates data indicative of joint angles of the portion of the body over the specific period of time.

30.-31. (canceled)

32. The method of claim 16 further comprising positioning the first and second markerless sensors.

33. The method of claim 32, wherein positioning the first and second markerless sensors comprises:
positioning the first markerless sensor in a fixed position relative to the body;
positioning the second markerless sensor in a temporary position relative to the body; and
iteratively altering the position of the second markerless sensor relative to the body by moving the second markerless sensor relative to the body and checking the accuracy of the estimates of at least one of joint positions and joint angles of the portion of the body over the specific period of time in the third set of data to determine an optimal position for the second markerless sensor.

34. The method of claim 33, wherein the accuracy is determined based on one or both:
a difference between the estimates in the third set of data and estimates determined using a marker-based system; and
a number of inferred positions and no positions in the first and second sets of data.

35. (canceled)

36. The method of claim 32, wherein positioning the first and second markerless sensors comprises:
positioning the first and second markerless sensors adjacent to each other relative to the body; and
iteratively altering the position of both the first and second markerless sensors relative to the body by moving both the first and second markerless sensors and checking the accuracy of the estimates of at least one of joint positions and joint angles of the portion of the body over the specific period of time in the third set of data to determine an optimal position for the first and second markerless sensors.

37. The method of claim 36, wherein the accuracy is determined based on one or both:
a difference between the estimates in the third set of data and estimates determined using a marker-based system; and
a number of inferred positions and no positions in the first and second sets of data.

38. (canceled)
